(19) United States
(12) Patent Application Publication
(10) Pub. No.: US 2004/0087026 A1
(43) Pub. Date: May 6, 2004

(54) Host cells for packing a recombinant adeno-associated virus (raav), method for the production and use thereof

(76) Inventors: Joan Bertran, Folgueroles (ES); Ulrich Moebius, Gauting-Unterbrunn (DE); Markus Horer, Martinsried (DE); Bernd Rehberger, Gersthofen (DE)

Correspondence Address: CLARK & ELBING LLP, 101 FEDERAL STREET, BOSTON, MA, 02110, US

(21) Appl. No.: 10/362,906

(22) PCT Filed: Sep. 7, 2001
(86) PCT No.: PCT/EP01/10370

(30) Foreign Application Priority Data
Sep. 8, 2000 (DE) 100 44 384.2

(51) Int. Cl. C12N 15/861; C12N 7/00; C12N 5/06

(52) U.S. Cl. 435/456; 435/457; 435/235.1; 435/325

(57) ABSTRACT

The invention relates to host cells for packing a recombinant adeno-associated virus (rAAV). Said cells contain at least one copy of a first auxiliary construct for expressing at least one AAV Rep protein, and at least one copy of another auxiliary construct for expressing at least one AAV Cap protein. The invention also relates to auxiliary constructs for expressing at least one AAV Rep protein and one AAV Cap protein in a host cell; vector constructs comprising at least one nucleic acid which is heterologous in relation to the AAV; a method for producing a host cell for packing a recombinant adeno-associated virus (rAAV); and the use of the host cell for producing an rAAV.

The present invention relates to a host cell for packaging a recombinant adeno-associated virus (rAAV) which comprises at least one copy of a first helper construct for stable expression of at least one AAV rep protein and at least one copy of another helper construct for stable expression of at least one AAV cap protein. The invention further relates to helper constructs for stable expression of at least one AAV rep protein and AAV cap protein in a host cell, to a vector construct which comprises one or more nucleic acid heterologous to AAV, and to methods for producing a host cell for packaging a recombinant adeno-associated virus (rAAV) and to the use of the host cell for producing rAAV.

Adeno-accociated virus (AAV) belongs to the parvovirus family. This virus family is characterized by an icosahedral, unenveloped capsid with a diameter of about 18-30 nm, which contains a linear and single-stranded DNA of about 5 kb. For efficient replication of AAV, especially of the subgroup of defective parvoviruses (dependoviruses), simultaneous infection of the host cell with helper viruses, for example with adenoviruses, herpesviruses or vacciniaviruses, is necessary. In the absence of a suitable helper virus, AAV enters a latency state, the viral genome being capable of permanent (=stable) integration into the genome of the host cell. This property of AAV makes it particularly interesting as transduction vector for mammalian cells. The two approx. 145 bp-long inverted terminal repeats (ITR) are generally sufficient for the vector function. They harbor the necessary signals for replication, packaging and integration into the host cell genome. For packaging in recombinant AAV particles (rAAV particles), a helper plasmid which harbors the genes for the nonstructural viral proteins (Rep proteins) and for the structural viral proteins (Cap proteins), and a vector plasmid which harbors the genes to be packaged, flanked by the AAV ITRS, is transfected into cells suitable for packaging, e.g. HeLa or 293 cells, which are then infected with a suitable helper virus. A lysate containing rAAV particles is obtained after some days.

The capsid of adeno-associated viruses consists naturally of the three proteins VP1, VP2 and VP3 in a proportion of 1:1:10. The AAV capsid genes are located at the right-hand end of the AAV genome and are encoded by overlapping sequences within the same open reading frame (ORF) using different start codons and one splice donor and two splice acceptor sites for expression thereof. The VP1 gene contains the entire VP2 gene sequence, which in turn contains the entire VP3 sequence with a specific N-terminal region. The fact that the overlapping reading frames code for all three AAV capsid proteins is responsible for the obligatory expression of all capsid proteins, although in different proportions.

Various AAV serotypes are known, of which the human AAV serotype 2 (AAV2) has been analyzed best. All the serotypes are virus vectors with advantageously properties for somatic gene therapy. The essential advantages are the stable integration of viral DNA into the cellular genome in the presence of the larger Rep proteins Rep 68 and Rep 78, the ability to infect cells which are not undergoing cell division (=resting cells), the stability of the viral capsid, which allows purification to high titers (10¹³-10¹⁴ particles per ml), the low pathogenicity, and the complete absence of viral genes and gene products in the recombinant AAV vector. It is precisely the last-mentioned aspect which explains why no cellular immune response to AAV-specific gene products is observed in in vivo applications, so that long-term expression (longer than one year) of therapeutic genes transferred with AAV is possible. This is why AAV vectors are very suitable in particular for use in gene therapy. For such a use there has been development in general of replication-defective viruses which, although they are able to infect a desired target cell and transfer the nucleic acid encoded in them to the cell, no longer themselves replicate in this cell. This is achieved for example by deleting genes which are important for viral replication, such as the genes encoding structural proteins, and, where appropriate, incorporating in their place the foreign nucleic acid to be transferred. For producing larger quantities, suitable for use in gene therapy, of viruses incapable of replication it is necessary for the deleted genes to be provided as so-called helper genes in “trans” in order to compensate for the defect in a virus no longer able to replicate in the cell.

To use AAV as viral transduction vector it is generally necessary to have larger quantities of recombinant AAV particles. A suitable method for producing these large quantities of rAAV particles is cotransfection of a eukaryotic cell with two recombinant AAV plasmids and subsequent infection with a helper virus (Chiorini J. A. et al. (1995) Human Gene Therapy, 6, 1531). The first rAAV plasmid (=vector construct) comprises the foreign nucleic acid which is to be transferred, and which is bounded, i.e. flanked, by the two ITR regions. The second recombinant AAV plasmid (=helper plasmid) comprises the AAV genes required to produce the particles (rep and cap genes). The absence of the ITR regions in the helper construct is intended to prevent the packaging of the rep and cap genes in the AAV particle and thus the development of unwanted wild-type AAV. Subsequently, suitable cells which are permissive, i.e. accessible, both for the recombinant AAV constructs and for a suitable helper virus are transfected with the two AAV constructs. Infection of the transfected cells (=producer cells) with, for example, adenovirus as suitable helper virus is followed by expression of AAV genes, replication of the transferred foreign nucleic acid and packaging, and the assembly of the recombinant AAV particles. The rAAV particles comprise the foreign nucleic acid, flanked on both sides by the ITR regions, in the form of single-stranded DNA. At the same time, the helper virus replicates in these cells, which in the case of adenoviruses as helper viruses ends after some days with lysis (=destruction) and, associated therewith, death of the infected cells. The resulting rAAV particles, and the helper viruses, are in this case partly released into the cell culture supernatant or remain attached to structures of the lysed cells. A review of the use of AAV as general transduction vector for mammalian cells is given, for example, by Muzyczka N. (1992) Current Topics in Microbiology and Immunology, 158, 97.

A considerable disadvantage in the production of rAAV particles is the concomitant development of replication-competent wild-type AAV (rcAAV) and the presence of helper viruses, for example adenovirus. However, with a view to safety on use of recombinant AAV particles for example in gene therapy, it is necessary for the preparations to be essentially free of helper viruses and wild-type AAV because, for example, adenoviruses as helper viruses lyse the infected cells and additionally cause cellular immune responses to adenoviral proteins. Moreover, adenoviruses are pathogenic for humans, causing nonspecific coryzal symptoms. Wild-type AAV may, in the presence of a helper virus, replicate and spread in the body. Moreover, under such conditions there would be expression of rep and cap genes which would in turn amplify rAAV genomes in the same cell and lead to new rAAV particles. This might lead to spread of rAAV genomes throughout the body.

It is possible with the aid of somatic gene therapy not only to correct genetic defects but also to transfer novel functions to normal or diseased cells as part of a therapy. Thus, to date a wide variety of vector systems have been developed in order to introduce foreign nucleic acids into a large number of cell types. Among the viral systems, AAV is understood to be the most promising vector system because it has a broad host cell tropism and integrates sequence-specifically preferentially into chromosome 19 (Kotin (1994) Human Gene Therapy 5, 793-801). It has thus recently been possible to show by means of various animal model systems that in vivo administration of rAAV leads to long AAV-mediated gene expression without also causing specific immune responses or inflammatory reactions in the successfully transduced cells (Daly et al. (1999) Human Gene Therapy 10, 85-94; Herzog et al. (1999) Nature Medicine 5, 56-63; Lo et al. (1999) Human Gene Therapy 10, 201-213; Snyder et al. (1999) Nature Medicine 5, 64-70).

Encouraged by the successes in the animal experiments described, HeLa-based packaging cell lines comprising copies of the entire AAV genome without the flanking ITRs and having the rep and cap genes under the control of the native viral promoters have been developed. The viral promoters P5, P19 and P40 are inactive in the absence of helper virus infection (Inoue and Russell (1998) J. Virol, 72, 7024-7031; Gao et al. (1998) Human Gene Therapy 9, 2353-2362). After helper virus infection, expression of AAV genes is induced in these stably transfected HeLa cells. The advantage of these cell lines is that rAAV particles can be produced on a large scale and reproducibly, which is necessary especially for a commercial production process (Allen et al. (1997) J. Virol. 71, 6816-6822; Wang et al. (1998) J. Virol. 72, 5472-5480).

One disadvantage of the use of these cell lines is the fact that only one recombination event with the vector genome is necessary for wild-type AAV to develop, which is why such cell lines are unsuitable for use in gene therapy because of the huge risk.

The present invention is therefore based on the object of providing host cells in the form of packaging and producer cells, and helper and vector constructs which are suitable for producing rAAV and which permit the production of rAAV on a large scale, but at the same time the development of wild-type AAV is essentially prevented.

It has now been found, surprisingly, that high yields of rAAV particles can be achieved with the aid of an HeLa-based packaging cell line in which the rep and cap genes of AAV are functionally separate, and the cap gene are under the control of the homologous promoters P5, P19 and P40, without, however, rcAAV particles being formed at the same time. It is possible in this connection for the functionally separate rep and cap genes to be present transiently, that is to say episomally, transfected, and to integrate at the same site, for example as concatemers, or at different sites in the cellular genome. The advantage is that at least two independent recombination events are necessary in each case for reconstitution of rcAAV particle.

During the experiments which were carried out there was use inter alia of a cap gene expression construct which, besides the promoter P40 which is absolutely necessary for expression of the cap gene, additionally comprises the regulatory sequences of the promoters P5 and P19. It was possible to establish in this connection that although strong expression of the Cap protein is achieved through cloning of the cap gene exclusively under the control of the promoter P40, this expression can no longer be regulated (=is constitutive) and thus cannot any longer be induced by helper viruses either. Such a cap expression construct which comprised only the promoter region of P40, but not of P5 and P19, was not stably integrated in the host cells. This phenomenon is explained by the toxicity of the constitutively expressed Cap protein for the host cell.

Thus, expression constructs for the Cap protein produced and used for the purposes of the present invention have at the promoter regions of the promoters P5, P19 and P40 modified by mutagenesis in such a way that although the promoter function remained intact in relation to the start of transcription, these constructs were unable to express any Rep protein. It was thus necessary for adenovirus-inducible trans-activation of the promoter P40 to provide the two large Rep proteins Rep 68 and Rep 78 from a second source (=in trans). It was then possible with the aid of such Cap expression constructs to achieve stable integration into the genome of the host cells. These constructs therefore have the advantage that, in the absence of a helper virus, no toxic amounts of Cap protein are expressed and, nevertheless, very strong Cap protein expression inducible by helper viruses is ensured.

One aspect of the invention is therefore firstly a host cell for packaging recombinant adeno-associated virus (rAAV) comprising at least one copy of a first helper construct for expression of at least one AAV Rep protein and at least one copy of a further helper construct for expression of at least one AAV Cap protein. In this case the nucleic acids coding for the Rep protein and the Cap protein are functionally separate from one another, and operatively linked to the natural AAV regulatory sequences. These are, in particular, the natural AAV promoters.

In a first preferred embodiment, the host cell additionally comprises at least one copy of a vector construct. Such a host cell is also referred to hereinafter as producer cell.

In a further preferred embodiment, the host cell additionally comprises at least one copy of a nucleic acid construct for at least one gene product of a helper virus and/or of a cellular gene which is necessary for producing rAAV.

The invention further comprises a helper virus-independent rAAV producer cell. This comprises, in addition to the producer cell, the genes, necessary for producing rAAV, of a helper virus and/or regulated cellular helper genes, so that these cells do not need to be infected with helper viruses to produce rAAV.

The operative linkage of the nucleic acids coding for the Rep protein and the Cap protein to the three natural AAV regulatory sequences, especially to the natural AAV promoters, results in large amounts of rAAV being obtained but, at the same time, the development of wild-type AAV being essentially prevented.

In a preferred embodiment, the adeno-associated virus (AAV) is selected from the serotypes AAV1, AAV2, AAV3, AAV4, AAV5 and/or AAV6. Likewise included according to the invention are capsid mutants of these serotypes. Capsid mutants mean for the purposes of this invention that the AAV particles may comprise a mutated capsid. This may comprise a mutation of one or more amino acids, one or more deletions and/or insertions. Corresponding examples are known to the skilled worker from the following references: WO 99/67393, Grifman M. et al. (2001) Mol Ther. 3(6): 964-75, Wu P. et al. (2000) J Virol 74(18): 8635-47, Chandler LA et al. (2000), Mol Ther. 2(2): 153-60, Hirate RK and Russell DW (2000) J. Virol. 74(10): 4612-20, Girod A et al. (1999) Nat Med. 5(12): 1438, Girod A et al. (1999) Nat Med. 5(9): 1052-6 or in Bartlett JS et al. (1999) Nat Biotechnol. 17(2): 181-6.

The terms “protein” and “polypeptide” are used synonymously for the purposes of the present invention and relate to a polymer of amino acids of any length. These terms likewise include proteins which have undergone post-translational modification steps such as, for example, glycosylation, acetylation or phosphorylation.

The terms “nucleic acid”, “DNA” and “polynucleotides” mean for the purposes of the present invention polymeric forms of nucleotides of any length, with the term relating only to the primary structure of the molecule. This term therefore encompasses single- and double-stranded DNA molecules just as much as modified polynucleotides such as, for example, methylated or protected (=capped) polynucleotides.

The terms “genes” and “gene sequences” refer to a polynucleotide which comprises at least one open reading frame and has the ability to produce a particular protein by transcription and translation.

The term “regulatory sequence” means a genomic region which regulates the transcription of a gene to which it is linked. Transcriptionally regulatory sequences as described in the present invention include at least one transcriptionally active promoter, but may also comprise one or more enhancers and/or terminators of transcription.

The term “operatively linked” refers to the arrangement of two or more components. Since the components are connected to one another, they are allowed to exercise their function in a coordinated manner. For example, a transcriptionally regulatory sequence or a promoter is operatively linked to the coding sequence if the transcriptionally regulatory sequence or the promoter respectively regulates or starts transcription of the coding sequence. A promoter operatively linked to a gene to be transcribed is generally referred to as “cis” element to the coding sequence, but it is not necessarily located in the direct vicinity of the gene to be transcribed.

The expressions “functionally independent units” or “functionally separate” mean that two or more genes do not overlap, where the term “gene” encompasses not only the coding sequence but also the corresponding promoter. Specifically, this means for the rep and the cap gene—for which in the wild-type AAV genome the coding sequence the rep gene overlaps with the coding sequence of the cap gene and with the cap promoter (p40)—that the two genes no longer overlap. This is achieved for example by both parts of the coding sequence used conjointly and the p40 promoter are duplicated (see, for example, FIG. 1A). This may mean different arrangements of the genes in the genome. One possibility is that the genes are located at different places in the genome, whether integrated at different sites into the genome or whether located on different plasmids or a mixture of these. A second possibility is for the genes also to be located side by side on the same DNA molecule, for example a chromosome or a plasmid, although each gene is controlled by its own promoter. An arrangement of this type is probable for example when two genes on different DNA molecules are transfected together. During the transfection, these molecules may form concatemers which then integrate at one site in the genome but still form functionally independent units.

The expression “recombinant” as used for the purposes of this invention refers to a genetic unit which is modified by comparison with the unit found naturally. Application of the term to an adeno-associated virus means that the virus harbors nucleic acid(s) which has/have been produced by a combination of cloning, restriction and/or ligation steps and which does/do not occur naturally in the adeno-associated virus.

The terms “natural promoter” and “homologous promoter” as used for the purposes of the invention mean that the genetic unit of the promoter or of the regulatory sequence is derived from the same organism as the remainder of the unit with which it is compared. Conversely, a “heterologous” or “unnatural promoter” means that the promoter has been separated from its natural coding sequence and has been operatively linked to another coding sequence.

“Stable expression” of a protein in a cell means that the DNA coding for the protein is integrated into the genome of the host cell and therefore is stably transmitted to daughter cells on cell division. “Stable expression” may also mean that the DNA is present episomally and is kept stable by independent replication. This is achieved for example by known, especially viral, replication systems consisting of an initiator protein (e.g. SV40 large T antigen, EBNA 1) and an origin of replication (e.g. SV40 ori, EBV orip). Although episomes such as, for example, plasmids may also under certain conditions be transmitted to the next generation, genetic material present episomally in the host cell is lost faster than is chromosomally integrated material. It is possible for example to incorporate the maintenance and transmission of the genetic material of interest through incorporation of a selectable marker in the direct vicinity of the polynucleotide of interest, so that host cells harboring the polylnucleotide can be kept under selection pressure.

The term “helper constructs” means for the purposes of the present invention recombinant AAV plasmids which comprise either the AAV rep genes and/or the AAV cap genes.

The term “vector construct” means according to the present invention a recombinant AAV plasmid which harbors foreign DNA (=transgene) bounded by ITR regions.

The term “packaging cell” means for the purposes of the present invention a host cell which comprises no vector construct although it comprises one or more helper constructs.

The term “producer cell” means for the purposes of the present invention a host cell which comprises both one or more helper constructs and one or more vector constructs. This producer cell may be helper virus-dependent if its infection with a helper virus is necessary for production of rAAV. It may, however, also be helper virus-independent if the cell harbors the genes necessary for inducing rAAV production, for example under the control of one or more inducible promoters, and thus does not need to be infected with helper virus for production of rAAV.

The term “vector cell” means for the purposes of the present invention a host cell which comprises no helper construct although it comprises one or more vector constructs.

One sort of helper constructs used to produce the host cell of the invention for packaging recombinant adeno-associated virus comprise nucleic acid sequences coding for at least one Rep protein, where Rep proteins mean the proteins Rep 78, Rep 68, Rep 52 and Rep 40, especially Rep 68, Rep 52 and Rep 40, in particular Rep 68 and Rep 52. The other helper constructs comprise nucleic acid sequences which code for at least one of the known Cap proteins, where the Cap proteins are the proteins VP1, VP2 and VP3. The genes for these proteins, and the ITR sequences, can be isolated from wild-type AAV which are generally obtainable in the form of clones. Thus, for example, the clone pSM620 is described by Samulski et al. (1982) Proc. Natl. Acad. Sci. USA, 79, 2077; the clone pAV1 is described by Laughlen et al. (1983), Gene 23, 65 and the clone sub201 is described by Samulski (1987) J. Virol. 61, 3096.

In a preferred embodiment of the present invention, the helper constructs for expression of the Rep protein and of the Cap protein are integrated as functionally independent units into the genome of the host cell. This effectively prevents formation of wild-type AAV (rcAAV) because, to form wild-type viruses, two recombination events would be necessary, which are quite rare, with a frequency of 10⁻⁷ each at each cell division, that is to say in total 10⁻¹⁴. In fact, no rcAAV was detectable in a recombinant virus preparation containing 2×10¹⁰ genomic particles.

In a further preferred embodiment, expression of the Rep protein is controlled by the natural AAV promoter P5 and expression of the Cap protein is controlled by the natural AAV promoter P40, in particular by the natural AAV promoters P19 and P40, especially by the natural AAV promoters P5, P19 and P40.

Foregoing experiments had shown that the use of heterologous promoters for Rep expression did not lead to appropriate regulation for a high yield of rAAV (Hölscher C. et al. (1994) J. Virol. 68, 7169-7177; Yang Q. et al (1994) J. Virol. 68, 4847-4856). In the most preferred embodiment of the present invention, the Cap expression plasmid comprises the AAV promoters P5, P19 and P40 in order to permit regulated expression depending both on helper virus infection or on helper virus gene products and on Rep protein expression, because this arrangement is the best reflection of the natural lytic AAV life cycle. This arrangement proved to be very suitable for strictly regulated Cap protein expression. Although numerous studies using heterologous promoters for expressing large amounts of Cap proteins have been published in the literature to date (Gao et al. (1998), supra, Grimm D. (1998) Human Gene Therapy 9, 2745-2760), for the purposes of this invention the natural promoters P5, P19 and P40 were used for Cap protein expression, because it was found that when the P40 promoter is no longer in the natural environment of the other promoters P5 and P19 it acts as constitutive promoter. Use of the natural AAV regulatory sequence made it possible to ensure that transcription factors required for regulated expression of the cap genes find all the binding sites in the natural promoter region in order to exercise their regulatory functions.

In a further preferred embodiment, expression of the Rep protein and of the cap protein in the host cell are regulated dependent on one another. This approach was chosen because it was found that in the first place weak Cap expression is necessary for efficient packaging of rAAV in stable cell lines because, otherwise, large Cap amounts have toxic effects on the cells. By contrast, strong Cap expression must take place at the time of packaging. A constitutive, heterologous promoter cannot comply with these two criteria simultaneously. Although this can be improved by using inducible, heterologous promoters, it is extremely difficult to implement accurate temporal regulation and the strength of Cap expression by such promoters in practice. The use of the natural homologous promoters couples the expression of Cap to the activation by helper virus gene products and/or cellular helper genes, and Rep, and thus provides temporal control exactly as in the wild-type situation.

Transcription of the nucleic acids coding for the rep proteins and the cap proteins is particularly advantageously terminated by the natural regulatory sequences, in particular by the natural AAV poly-A signal. The use of the homologous sequences for terminating transcription of the AAV cap and rep genes increases, in a similar way to the initiation of transcription, the amount of rAAV particles produced by the AAV vector system.

It is suitable to use as host cell a mammalian cell, in particular a human cervical carcinoma cell, especially an HeLa cell. HeLa cells have proved to be particularly advantageous because the AAV P5 promoter is virtually inactive in HeLa cells, and it is therefore possible for an expression cassette for the AAV Rep protein to be stably integrated into their genome under the control of the natural regulatory elements, so that the Rep protein has no toxic effect in these cells (Clarke et al. (1995) Human Gene Therapy 6, 1229-1341; Tamayose et al. (1995) Human Gene Therapy 7, 507-513; Inoue & Russell (1998) supra, Gao et al. (1998) supra).

Further aspects of the present invention relate to a first helper construct for stable expression of at least one AAV Rep protein in a host cell, where the nucleic acid coding for the Rep protein is operatively linked to the natural regulatory sequences of AAV, especially to the natural AAV promoters P5 and P19, and to a second helper construct for stable expression of at least one AAV Cap protein in host cell, where the nucleic acid coding for the Cap protein is operatively linked to the natural regulatory sequences of AAV, preferably to the natural AAV promoter P40, in particular to the natural AAV promoters P19 and P40, especially to the natural AAV promoters P5, P19 and P40.

The advantage of separating rep and cap into different expression units is that it is possible by replacing the cap gene or the cap expression unit by a cap gene or a cap expression unit of a different AAV serotype to generate a AAV particle of a different serotype, it being possible to use the same rep gene or the same rep expression unit and the same vector construct. This therefore minimizes the effort if it is intended, for a wide variety of reasons, to use different AAV serotypes of the same vector construct.

A further aspect of the present invention is nucleic acid sequences comprising a helper construct and coding for at least one Rep protein, where the Rep proteins are Rep 68, Rep 52 and/or Rep 40, but not Rep 78, because it has surprisingly been possible to establish that, besides Rep 52, Rep 40 and the three Cap proteins VP1, VP2 and VP3, additional expression only of Rep 68 is sufficient for packaging of AAV vectors. The advantage of these Rep 78-deficient helper constructs is that the largest Rep protein, which is usually toxic for the packaging cells, is not expressed at all. It has been found further that, among the Rep proteins, Rep 78 has the greatest inhibitory activity on cellular processes such as, for example, transcription. It is therefore possible on use of this helper construct, because of the absence of Rep 78, to increase the packaging efficiency. Both Rep 68 and Rep 78 are expressed by the p5 promoter in the natural system. A further reason why the use of the Rep 78-deficient helper construct is advantageous is because Rep 68 Represents the stronger transactivator, compared with Rep 78, of the AAV promoters P19 and P40 in adenovirus-infected cells (Horer et al. (1995) J. Virol. 69, 5485-5496; Weger et al. (1997) J. Virol. 71, 8437-8447). Use of this Rep 78-deficient helper construct therefore leads to increased expression of the smaller Rep proteins Rep 40 and Rep 52, and of the capsid proteins and thus of the desired higher packaging efficiency.

The Rep 78-deficient helper construct pUC“Rep68,52,40Cap” (RBS)Δ37 (cf. FIG. 12) was produced by cloning the AAV sequences from nucleotide 201 to nucleotide 4497, including deletion of the intron sequence, and from nucleotide 658 to nucleotide 4460 into the bacterial expression plasmid pUC19, the binding sites for the Rep protein in the pUC19 sequence having been deleted (cf. FIG. 6). Through use of this strategy, two rep genes and at least two cap genes, each with its own poly(A) sequence for terminating transcription, are arranged consecutively. Starting from the first section (AAV sequence nucleotide 201 to nucleotide 4497) it is possible to express the Rep proteins Rep 68 and Rep 40, and the Cap proteins VP2 and VP3, while starting from the second section (AAV sequence nucleotide 658 to nucleotide 4460) the Rep proteins Rep 52 and Rep 40, and the Cap proteins VP1 VP2 and VP3, are expressed. Overall, therefore, all the AAV-2 proteins with the exception of Rep 78 are encoded.

The likewise Rep 78-deficient helper construct pUC“ΔRep78Cap” (RBS)Δ37 (cf. FIG. 13) was produced by likewise cloning said AAV sequences (nt 201-2310; nt 658-4460 including deletion of the intron sequence) into the bacterial expression plasmid pUC19 (cf, FIG. 6). Once again, the binding sites for the Rep protein in the pUC19 sequence was deleted. The rep gene was partially duplicated in this way. The helper construct produced in this way contains only one poly(A) sequence, so that all mRNA transcripts have the same 3′ end. Starting from the first section (AAV sequence nucleotide 201 to nucleotide 2310) it is possible to express the Rep proteins Rep 68 and Rep 40, while starting from the second section (AAV sequence nucleotide 658 to nucleotide 4460) the Rep proteins Rep 52 and Rep 40, and the Cap proteins VP1, VP2 and VP3, are expressed. Overall, therefore, all the AAV-2 proteins with the exception of Rep 78 are also encoded by this vector construct.

A helper construct pUC“ΔRep78ΔCap” (RBS)Δ37 for expressing the Rep proteins Rep 68, Rep 52 and Rep 40 was produced by deleting the AAV nucleotides 2945 to 4046 from the cap gene (nucleotides 2203 to 4410) of the helper construct pUC“ΔRep78ΔCap” (RBS)Δ37. Functional Cap proteins can no longer be expressed owing to this deletion.

A further aspect of the present invention are vector constructs comprising one or more nucleic acids which are heterologous to AAV and which are flanked by ITR sequences, with the 5′-located ITR sequence having a deletion in the region of the C palindrome.

These vector constructs comprise the AAV sequences 1-60/83-191 (ΔC arm ITR as left ITR—see below for explanation thereof) and 4498 to 4671 (as right ITR).

It is known that the ITR sequences of AAV-2, which are 145 base pairs long, are composed of a large palindrome (A) and two smaller palindromes (B and C). The first 125 bases of the ITR sequence for a T-shaped hairpin structure (see, for example, Muzyczka, N. (1992), supra). It is possible in this case for the terminal sequence to be in one of two configurations. In the first configuration, also called “flip”, the B palindrome is nearer to the 3′ end, and in the second configuration, also called “flop”, the C palindrome is nearer to the 3′ end (see also Svivstava, A. et al. (1983) J. Virol, 45(2), 555). The two configurations frequently change their configuration through a recombination event, which brings about destabilization of the corresponding vector constructs.

It has now been found, surprisingly, that a deletion within the 5′-flanking ITR sequence, which comprises in a preferred embodiment 80 nucleotides, in particular 40 nucleotides, especially 22 nucleotides, in the region from nucleotide 61 to 82, brings about the stabilization of these vector constructs, because a change of configuration between flip-flop orientations is no longer possible (cf. FIG. 8).

It has further been found, surprisingly, that vector constructs which have such a deletion in the C palindrome of the 5′ ITR sequence can be packaged just as efficiently as vector constructs with intact ITR sequences.

The present invention therefore relates further to a vector construct comprising one or more nucleic acids which are heterologous to AAV and which are flanked by ITR sequences, the 5′-located ITR sequence having a deletion in the region of the C palindrome.

A further aspect of the present invention is furthermore a vector cell comprising a vector construct where the 5′-located ITR sequence has a deletion in the region of the C palindrome.

A further aspect of the present invention relates to a vector construct comprising one or more nucleic acids which are heterologous to AAV, in particular a nucleic acid coding for a protein selected from a cytokine, in particular IL2, IL4, IL12 and/or GM-CSF (granulocyte macrophage colony stimulating factor) and/or a costimulating molecule, in particular B7, especially B7.1 and/or B7.2. However, it is possible according to the present invention to use as heterologous nucleic acid sequence any coding or else noncoding nucleic acid sequence. Preferably, one or more heterologous nucleic acid sequence(s) is introduced into a replication-defective vector construct by conventional cloning techniques known to the skilled worker (Sambrook et al. (1989) Molecular Cloning: A Laboratory Manual, Cold Spring Harbor Laboratory, Cold Spring Harbor, N.Y.).

Further suitable nucleic acid sequences are coding sequences for chemokines such as lymphotactin, Rantes, MCP-1 or Mip 1α, cytokines such as IL12, IL7, IL18, IL2, GM-CSF, IL1, IL6, interferon γ or IL-10, or antibodies, antibody fragments or single-stranded antibodies, for example directed against ICOS, also against the ICOS receptor, CD40, CD40 ligands, VEGF, IL-1, TNF-α, against tumor antigens such as, for example, Her-2/new, GD3 or CA125, against viral antigens or against IgE; furthermore against soluble receptor forms such as ICOS FC, ICOS-ligand FC, CD40L FC, TNF-α receptor FC, against apoptosis-inducing molecules such as proteins of the BCL-X family, BAX, BAD or caspases, necrosis-inducing peptides such as performs, toxins, for example derived from bacteria, viral tumor antigens such as HPV E6 or E7, nonviral tumor antigens such as B lymphoma-specific idiotype antibodies, BCRA-1, CA-125, α-fetoprotein, CEA, p53, and coagulation factors such as factor VIII or factor IX.

Noncoding sequences are further suitable for use as ribozymes or antisense RNAs.

WO 98/06746 discloses that a genetically manipulated melanoma cell line which expresses GM-CSF can be used as vaccine. WO 94/16716 discloses the use of a recombinant virus in cancer therapy, with use of at least one cytokine, for example GM-CSF or B7 and/or one tumor-associated antigen. The B7 gene refers in this case to the so-called B7.1 gene. WO 94/04196 discloses a DNA construct for treating oncoses which codes for a cytokine and, in addition, for B7, where this also means B7.1. WO 92/00092 discloses a nucleic acid sequence coding for B7.1, WO 94/03408 and WO 95/06738 disclose a nucleic acid sequence coding for B7.2, and EP-B1-0 188 479 discloses a nucleic acid sequence coding for GM-CSF.

The use of B7.2 in conjunction with the vector construct is particularly advantageous because in vitro investigations have shown that, in contrast to interferon γ or IL12, the B7.2 protein in association with B7.1 has an inhibitory effect on the activation of T lymphocytes (Rudy et al. (1997) Int. Immunol., 9, 853). It is possible in the presence of a second nucleic acid which codes for GM-CSF to increase the oncolytic effect of a B7 molecule (it being possible to use B7.1 or B7.2 here).

A further aspect of the present invention is therefore said vector construct comprising one or more nucleic acids which are heterologous to AAV, in particular a nucleic acid coding for a protein selected from a cytokine, in particular IL2, IL4, IL12 and/or GM-CSF and/or a costimulating molecule, in particular B7, especially B7.1 and/or B7.2. Particular preference is given, because of the relative ease of handling, to so-called double vectors which comprise both the nucleic acid sequence coding for GM-CSF and the nucleic acid sequence coding for B7 (cf. FIG. 7). The use of double vectors reduces in particular the number of packaging operations. Surprisingly, the double vectors of the invention permit comparably efficient expression of the two foreign nucleic acids as does coinfection with two individual vectors (cf. likewise FIG. 7). In particular, the heterologous nucleic acids are flanked by AAV ITR sequences, and expression thereof is regulated by a promoter and/or enhancer heterologous to AAV, in particular by the major immediate early enhancer/promoter (MIEP) of cytomegalovirus (CMV). Suitable promoters are all promoters which are heterologous to AAV and which are active in eukaryotic cells, preferably mammalian cells. Examples thereof are the SV40 promoter (Samulski et al. (1989) J. Virol, 63, 3822), the CMV enhancer/promoter mentioned (Vincent et al. (1990) Vaccine, 90, 353) or the LTR promoter of retroviruses (Lipkowski et al. (1988) Mol. Cell. Biol. 8, 3988). However, the CMV MIEP is particularly preferred because of its very strong expression, which is subject to only minimal variations.

A further aspect of the present invention is a method for producing a host cell for the packaging and/or production of recombinant adeno-associated virus (rAAV), which comprises the steps:

(a) production of a Rep helper construct and of a cap helper construct,

(b) introduction of the Rep helper construct into a host cell,

(c) selection of a host cell comprising the Rep helper construct,

(d) introduction of the Cap helper construct into the selected host cell and

(e) selection of a packaging cell comprising the Rep helper construct and the Cap helper construct.

The sequence of the introduction of Rep helper construct and Cap helper construct can also be reversed. It is likewise possible for the introduction of a Rep helper construct and of a Cap helper construct to take place at essentially the same time.

A further possibility is to produce a helper virus-independent packaging cell of rAAV, comprising the additional steps of

(e/d) introduction of at least one helper gene of the helper viruses and/or one regulated cellular helper gene into the producer cell

(f/e) selection of a vector cell comprising the helper gene(s).

A cell of this type would have the advantage that no helper virus infection is necessary for rAAV production; on the contrary, rAAV production could be initiated for example by inducing the promoters of the helper genes. Helper genes mean in this connection the genes of the helper viruses of AAV and/or cellular genes whose gene products are necessary for AAV replication or promote the latter. In adenoviruses for example, the helper genes are E1A, E1B, E4, E2A and VA. E1A is necessary for transactivation of the AAV p5 promoter. The E1B and E4 gene products serve in this connection to enhance AAV mRNA accumulation. The E2A and VA gene products serve to enhance AAV mRNA splicing, and translation. Also included as helper genes according to the invention are herpes simplex virus (HSV) helper genes. In a preferred embodiment, these are the 7 replication genes UL5, UL8, UL9, UL29, UL30, UL42 and UL52. UL5, 8 and 52 form the HSV helicase-primase complex, UL29 codes for the single-stranded DNA binding protein, UL42 for a double-stranded DNA binding protein, UL30 codes for the HSV DNA polymerase and, finally, UL9 codes for a protein which binds the HSV origin of replication (see Weindler F W and Heilbronn R (1991) J. Virol. 65.(5): 2476-83). Use of the helper virus in place of the individual helper genes, for example the adenovirus type 5 (Ad5), is particularly advantageous because this comes closest to the natural situation of AAV replication in the presence of helper viruses, and thus the packaging of rAAV particles is very efficient. Examples of other helper viruses are herpes viruses or vaccinia viruses.

One component of the invention is the production of a rAAV vector cell, comprising the steps:

(a) production of a vector construct,

(b) introduction of the vector construct into a host cell and

(c) subsequent selection of the vector cell comprising the vector construct.

A further possibility is to produce a helper virus-independent rAAV vector cell, comprising the additional steps

(d) introduction of at least one helper gene of the helper viruses and/or one regulated cellular helper gene into the producer cell

(e) selection of a vector cell comprising the helper gene(s).

The advantages of this helper virus-independent vector cell correspond to those of the helper virus-independent packaging cell.

A further aspect of the present invention relates to a method for producing a rAAV producer cell, which comprises the steps:

(a) production of a packaging cell as described above

(b) introduction of at least one vector construct into the packaging cell

(c) selection of a producer cell comprising the vector constructs).

A further possibility for producing a producer cell consists of the steps:

(a) production of a vector cell, as described above

(b) introduction of at least one helper construct described above into a vector cell; with a plurality of helper constructs this can take place together or sequentially,

(c) selection of a producer cell comprising the helper construct(s).

A further aspect of the invention relates to the production of a helper virus-independent rAAV producer cell, which comprises the additional steps:

(d) introduction of at least one helper gene of the helper viruses and/or one regulated cellular helper gene into the producer cell

(e) selection of a producer cell comprising the helper gene(s).

The advantages of this helper virus-independent producer cell correspond to those of the helper virus-independent packaging cells.

Introduction of constructs generally means transfection for the purposes of this invention. It is moreover possible for the construct not to be permanently integrated into the genome of the host cell, which is generally referred to as transient transfection. As an alternative to this, however, the construct, in particular the vector construct, can also be stably integrated into the genome of the host cell or be retained as stable episomal copy (by means of a replication system for example from SV40 large T antigen/SV40 ori or EBNA 1/EBV orip). Such integrated constructs are replicated in accordance with the DNA replication of the host cell genome, and subsequently transmitted to the daughter cells.

In a particular embodiment of the method of the invention, the introduction of the construct/constructs, in particular of the vector construct(s), takes place by infection with viruses. Preference is given in this connection to recombinant viruses, it being possible to use for example rAAV, adenoviruses, herpesviruses, vacciniaviruses, baculoviruses and/or phages, especially bacteriophages.

A preselection for cells with integration events is furthermore appropriate for the abovementioned method for the production of packaging, vector and/or producer cells for selecting cells with stably integrated constructs. For example, the Rep or Cap helper construct to be transfected can be mixed with a reporter construct in the ratio 10:1 and introduced, for example cotransfected, jointly into the host cell. This is followed by selection for the reporter, because it can be assumed that the cells in which the reporter construct has integrated have also, with high probability, integrated the respective helper construct.

The appropriate cells can in each case be selected by detection of protein expression, for example by means of Western blotting. Quantitative detection of a construct-specific nucleic acid in the cells would likewise be suitable, for example by a quantitative polymerase chain reaction (PCR), or Southern or Northern blotting.

A preferred way of detecting suitable cells is also direct detection of the packaging of rAAV in the these cells, by introducing constructs which are lacking for the packaging into the particular cell, and initiating the packaging by infection with a helper virus.

For the packaging cell comprising the Rep or Cap helper construct there is a requirement in each case for the other helper construct, and for a vector construct for example having a color marker such as GFP as transgene. By contrast, only a vector construct is necessary for a packaging cell having Rep and Cap helper constructs. Rep and Cap helper constructs are required for a vector cell. Neither a helper nor a vector construct is necessary for a producer cell. These cells can subsequently be induced to produce rAAV by introduction of the viral helper genes, in particular by infection with a helper virus. In contrast with this, the helper virus-independent packaging, vector of producer cells do not require introduction of helper genes as long as they already comprise all the necessary helper genes, or require only the introduction of a few helper genes. For these cells it is merely necessary to induce the helper genes in order to start rAAV production.

The AAV titer can subsequently be determined by conventional methods and then serves as a measure of optimal Cap or Cap/Rep expression. This procedure has the advantage that in the selection of the appropriate cells there is selection not only for absolute quantities expressed but also for the optimal ratio of Cap to Rep expression. This method moreover has the advantage that, in this case, there is additional selection for intact Rep and Cap genes, because Rep and/or Cap mutants which do still produce protein which is, however, no longer suitable for packaging of rAAV would not be identified in the other detection methods mentioned.

A further aspect of the invention relates to the use of a Rep helper construct and/or of a Cap helper construct and/or of a vector construct, and/or of a packaging cell, of a vector cell or of a producer cell, in particular a helper virus-independent producer cell, for producing rAAV.

The rAAV vector constructs comprise one or more nucleic acids which are heterologous to AAV. In the case where, as already described above, GM-CSF and B7.1 and/or B7.2 are used as heterologous nucleic acid, the rAAV particle which results from the packaging operation and which harbors the two immunostimulatory genes can be used as efficient transduction vector for the treatment of various oncoses, for example melanoma or ovarian carcinoma.

The figures and the examples which follow are intended to explain the invention in more detail without, however, restricting it.

### DESCRIPTION OF THE FIGURES

FIG. 1A shows a diagrammatic representation of some helper constructs of the invention compared with wild-type AAV. The natural AAV promoters P5, P19 and P40 are shown, as is the major intron of the AAV genome (“I”) and the natural poly(A) signal of the AAV genome (“pA”). The coding sequences are also shown for the rep gene and the cap gene of AAV.

FIG. 1B shows a diagrammatic representation of three different vector constructs, showing the ITRs at the left and right flanking end. The designation “CMV” stands for the major early promoter of cytomegalovirus. The designation “I” indicates an intron present in the pCI plasmid from Promega. The designation “pA” indicates the poly(A) signal of simian virus 40 (SV40). The abbreviation “GFP” stands for green fluorescent protein, “B7.2” for the immunocostimulatory protein B7.2 “GM-CSF” for granulocyte/macrophage colony stimulating factor, and “nLacZ” stands for the nuclear-localized form of the enzyme β-galactosidase.

FIG. 2 depicts the identification of the stable Rep cell lines by a Western blotting experiment. HeLa cells were transfected with the Rep helper construct (P5 Rep), with Rep expression being controlled by the natural AAV promoters P5 and P19. The hygromycin selection marker which was transfected in the ratio 1:19 to the Rep helper construct shown is not depicted. Subsequently, several hygromycin-resistant cell clones were picked, and Rep expression was induced with adenovirus as helper virus (lane 1-8). The entire quantity of cellular protein was isolated and analyzed by Western blotting using a specific Rep antiserum. The numbers 78/68/52/40 refer to the Rep proteins rep 78, Rep 68, Rep 52 and Rep 40. “M” stands for a molecular weight standard, “+” for positive control and “−” for negative control.

FIG. 3 shows the inducible Cap gene expression starting from various constructs. The Cap helper constructs depicted in the lower zone were used to transfect the Rep-expressing cell line R84. The cells were subsequently infected with adenovirus (MO15) (lanes 1 to 4 and wt labeled with “+”) or not infected as control (lanes 1 to 4 and wt labeled with “−”. After 72 hours, the entire cellular protein content was investigated by Western blotting analysis for expression of the three Cap proteins VP1, VP2 and VP3.

FIG. 4 shows the inducible expression of the Rep and Cap proteins in the stable packaging cell line C97 after adenovirus infection. C97 cells or HeLa control cells were either infected with adenovirus (MO15) or not infected as control. 72 hours after the infection, the entire cellular protein was extracted and analyzed by Western blotting for the presence of the Cap and Rep proteins using antibodies specific therefor. After adenovirus infection, the four Rep proteins Rep 78, Rep 68, Rep 52 and Rep 40 are clearly evident, and to a smaller extent the Cap proteins VP1 and VP2, and to a larger extent the Cap protein VP3, are clearly evident in the packaging cell line C97.

FIG. 5 shows the replication of recombinant AAV (rAAV) in C97 cells, with the rAAV replication intermediates being detected as low molecular weight DNA isolated from C97 cells previously infected with rAAV and adenovirus (MO15). It was typically possible to observe a complete cytopathic effect 72 hours after the infection, and the cells were collected at this time. Half of the cells were used to isolate low molecular weight DNA (first amplification round), and the other half of the cells was frozen and thawed again three times, and then centrifuged, in order to remove cellular constituents. The supernatant was then treated at 56° C. for 30 min in order to inactivate intact helper viruses. The supernatant was subsequently used to infect fresh C97 cells together with new helper viruses (MO15). A distinct cytopathic effect was again observable, and low molecular weight DNA was isolated (second amplification round). The DNA isolated from the first and second amplification round was analyzed by Southern blotting, using the vector construct of the invention as probe for the presence of the replication intermediates of the AAV genomes, in order to obtain an infectious titer of an original AAV stock.

FIG. 6 shows diagrammatically further helper constructs, all of which are derived from the cloning vector pUC19.

FIG. 7 shows diagrammatically the single and double expression vectors.

FIG. 8 shows diagrammatically the 5′-located ITR sequence of the AAV vector constructs having the configurations flip, flop and deletion of the C palindrome, which is referred to as “A(C arm)”.

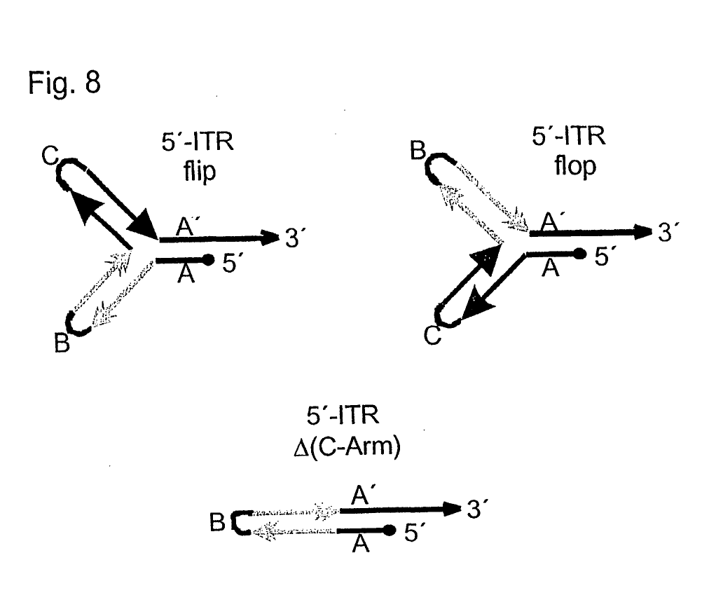

FIG. 9 shows diagrammatically the nucleotide sequence of the 5′-located ITR sequence of the various configurations from FIG. 8.

FIG. 10 shows the nucleotide sequence of the 5′-located ITR sequence with and without deletion, and of the 3′-located ITR sequence.

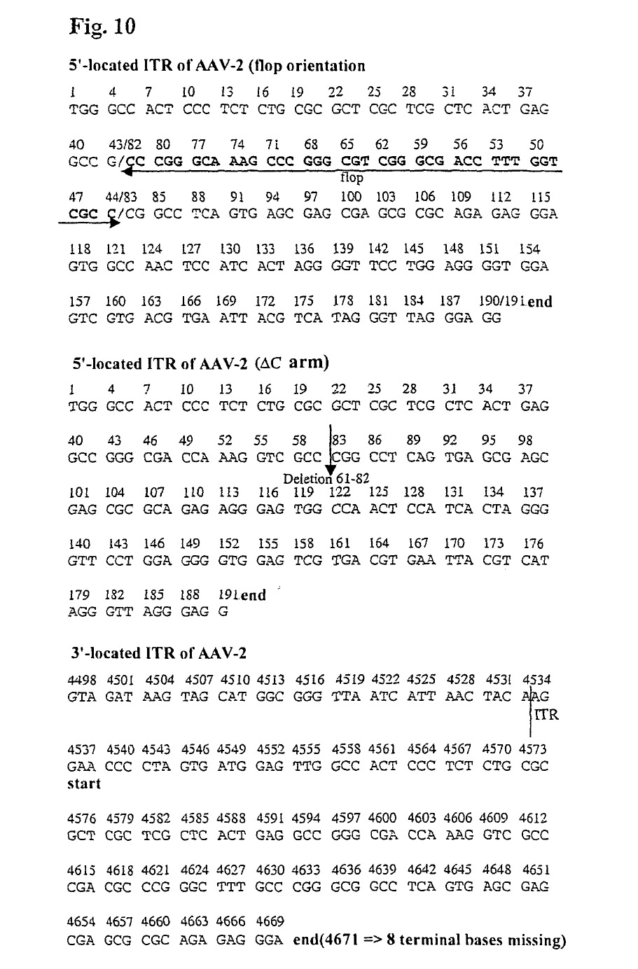

FIG. 11 shows diagrammatically the equally efficient packaging of a vector construct with deleted 5′-located ITR sequence [pAAV-(B7.2free+GM-CSF)] compared with a vector construct with a 5′-located ITR sequence in the flop orientation configuration [pAAV-(B7.2+GM-CSF)].

FIG. 12 shows diagrammatically a Rep 78-deficient helper plasmid referred to as pUC“Rep68,52,40Cap” (RBS)Δ37.

FIG. 13 shows diagrammatically a further Rep 78-deficient helper plasmid referred to as pUC“ΔRep78Cap” (RBS)Δ37.

### EXAMPLES

1. Plasmid Construction

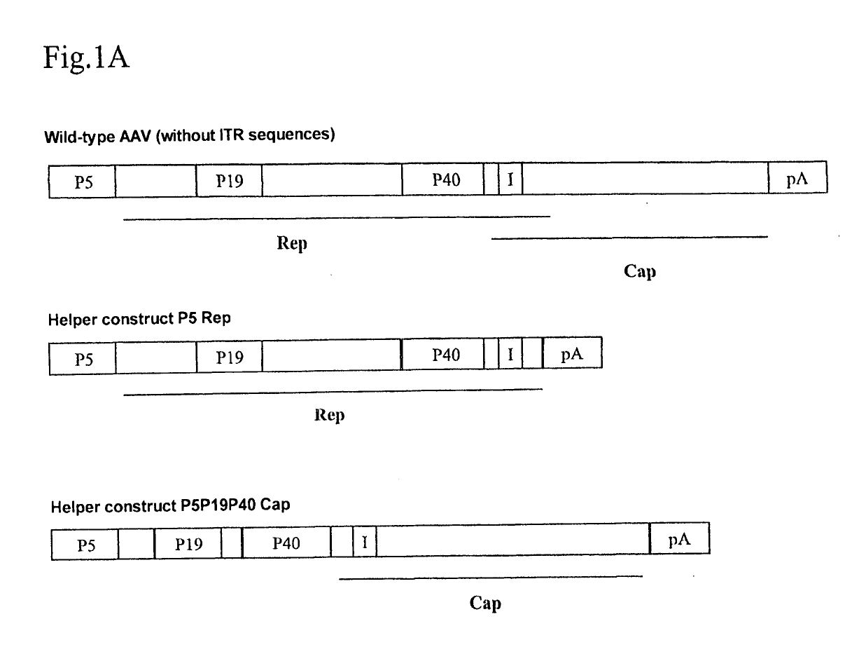

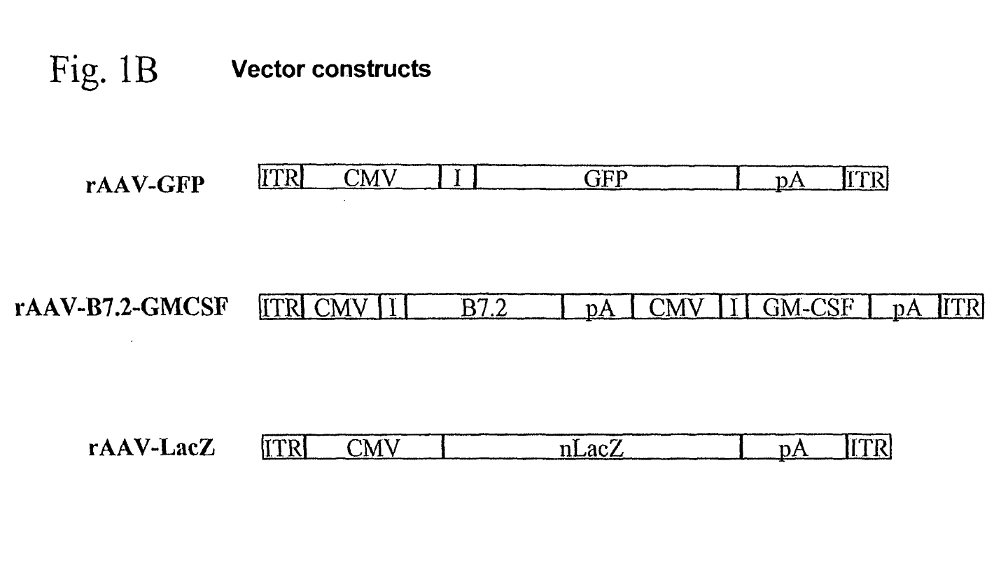

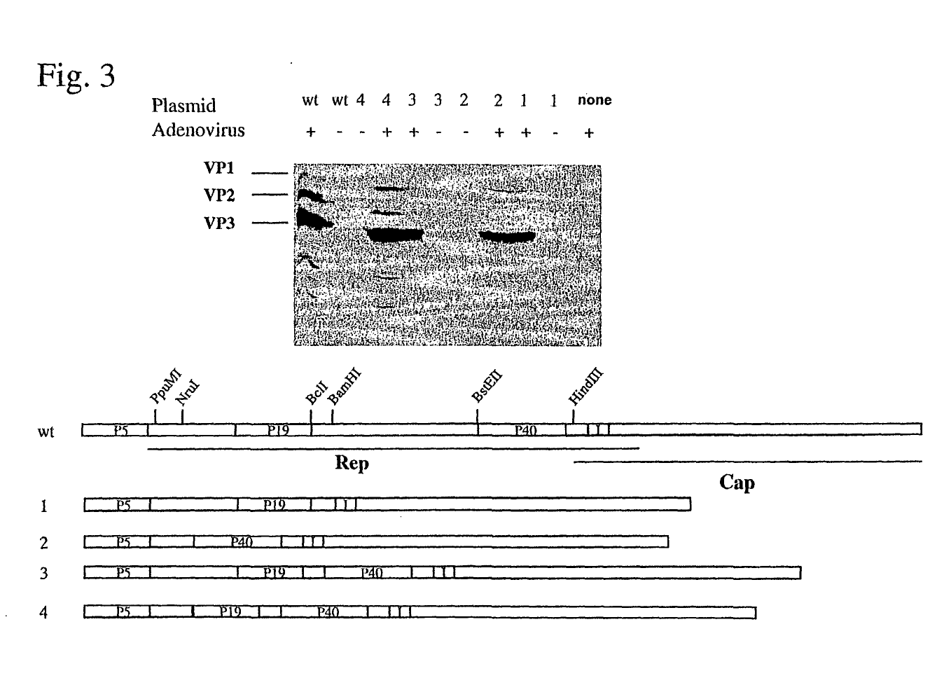

The plasmids (vector construct, helper constructs) used for the purposes of the present invention were produced applying standard cloning techniques which can be referred to in Sambrook et al. (1989), supra. The functionally relevant sections of these plasmids are shown in the diagrammatic representations in FIGS. 1 to 3, 6, 7, 12 and 13. The plasmid P5 Rep (FIG. 1A) was produced by deleting a DNA fragment which comprised nucleotides 2300-4170 of the AAV genome. (Ruffing et al. (1994) J. Gen. Virol. 75, 3385-3392 (Gene Bank Accession No. AF 043303). P5 RepΔ37 was obtained by deleting the AAV bases 4461-4497 from P5 Rep. The plasmid P5P19P40Cap (FIG. 1A) was obtained by deleting the DNA section between nucleotides 350 to 650 and 1045 to 1700. P5P19P40CapΔ37 was obtained by deleting the AAV bases 4461-4497 from P5P19P40Cap. Plasmids 1-4 shown in FIG. 3 contain various deletions of the AAV genome. Plasmid 1 lacks the sequence between the BclI cleavage site and the HindIII cleavage site of the AAV2 genome. Plasmid 2 lacks the sequence between the NruI cleavage site and the BstEII cleavage site, plasmid 3 lacks the sequence between the BamHI cleavage site and the BstEII cleavage site. Plasmid 4 corresponds to the plasmid P5P19P40Cap from FIG. 1A. The vector constructs for rAAV-B7.2-GM-CSF and rAAV-GFP were constructed using the pCI plasmid from Promega (Germany) and then transferred into a pUC19-based plasmid having the ITR sequences (cf. PCT/EP00/01090). The vector construct nLacZ which was likewise used as control has already been described in the literature (Bertran et al. (1996) J. Virol. 70, 6759-6766).

2. Packaging of Various Vector Constructs

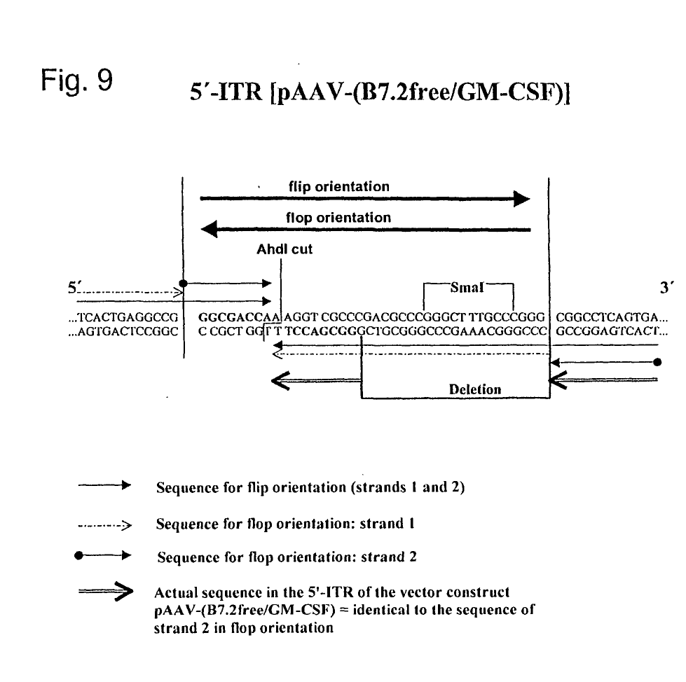

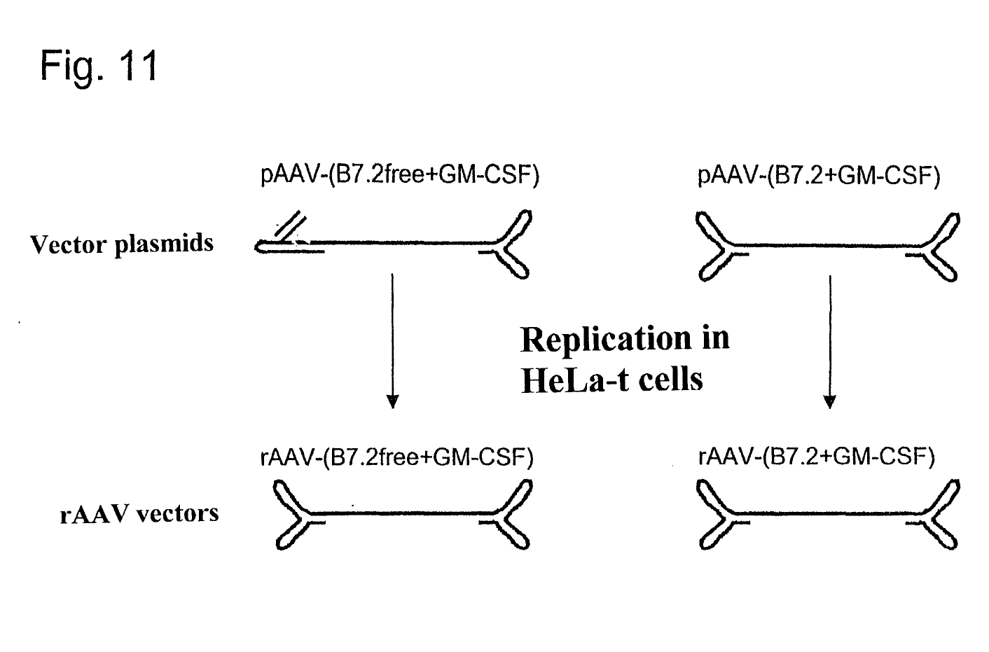

In order to investigate the influence of the deletion of the C palindrome in the 5′-located ITR of particular vector constructs on the expression of the foreign DNA (=transgenes), and the packaging of these vector constructs into rAAV, comparative transfection and packaging experiments were carried out. For this purpose, several clones of a vector construct were transfected with intact 5′-ITR (for example pAAV-(B7.2/GM-CSF)—cf. FIG. 11) and clones of a vector construct with a deletion in the C palindrome of the 5′-ITR (AC arm—for example pAAV-(B7.2free/GM-CSF)—cf. FIGS. 9 and 11)) into HeLa-t cells, using 6 μg of plasmid in each case for 4×10⁵ HeLa-t cells. 40 hours after the transfection, both the proportion of B7.2-expressing cells was found by FACS analysis, and the expression of GM-CSF in the culture supernatant was found by an ELISA technique. The values indicated in the table represent averages of duplicates of in each case 4 different clones of the same plasmid type. In the first experiment, for unknown reasons, the transfection rate achieved was only about a factor of one hundred less than the efficiency of the second experiment. This explains why the values found for GM-CSF in the cell culture supernatants in experiment 1 were two logarithmic units lower than in experiment 2. It was thus possible to transfect only about 40% of the cells in experiment 1, whereas virtually all the cells in experiment 2 expressed the protein B7.2.

TABLE 1

| | | B7.2 | GM-CSF |
|---|---|---|---|
| | | (% positive | (ng/E6 |
| Vector plasmid | Experiment | cells) | cells/48 h) |
| pAAV-(B7.2(GM-CSF) | 1 | 40.3 | 128 |
| pAAV-(B7.2free/GM-CSF) | 1 | 42.6 | 133 |
| pAAV-(B7.2/GM-CSF) | 2 | 95.4 | 12000 |
| pAAV-(B7.2free/GM-CSF) | 2 | 93.2 | 11000 |

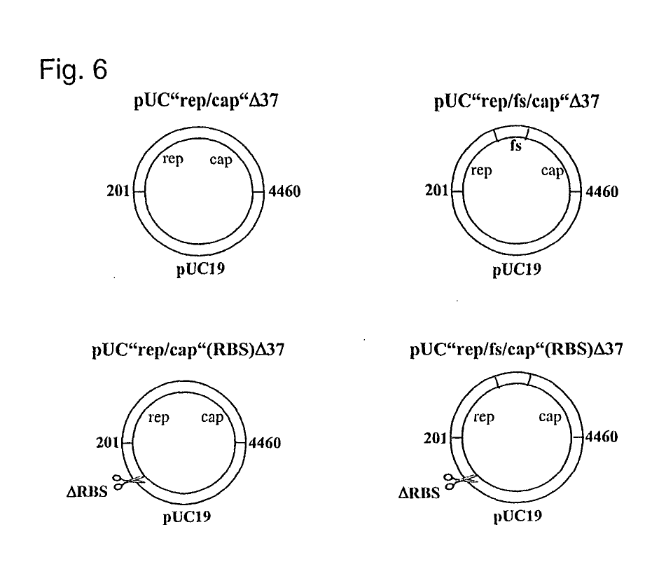

As is clearly evident from table 1, the transfection efficiencies achieved with the two vector plasmids are equally good, so that the deletion in the C palindrome of the 5′-located ITR has no negative effect on the strength of expression of the expression cassettes, cloned between the ITRs, for the transgenes. Subsequently, the same vector plasmids were used to carry out experiments on packaging in rAAV. For this purpose, in each case 4 μg of vector plasmid were transfected with 12 μg of helper plasmid (pUC“rep/cap” (RBS)Δ37—cf. FIG. 6) into 10⁶ HeLa-t cells. It was likewise observed in this case—as already described above, that the transfection efficiencies varied by two logarithmic units. After two days, the cells were infected with Ad-5 (MO12). Three days after the infection, the cells were disrupted in 3 ml of cell culture medium by repeated freezing and thawing, the cell constituents were pelleted by centrifugation, and the rAAV lysate was incubated at 60° C. for 10 min to inactivate helper viruses. 500 μl of lysate in experiment 1 (low transfection rate) and 5 and 25 μl of lysate in experiment 2 were used to infect 3×10⁵ irradiated (100 Gy) HeLa-t cells. 40 hours after the infection, both the proportion of B7.2-expressing cells was found by FACS analysis, and the expression of GM-CSF in the cell culture supernatant was found by an ELISA technique. The values indicated in table 2 below represent averages from duplicates of in each case 4 different clones of the same plasmid type:

TABLE 2

| | | | GM-CSF |
|---|---|---|---|
| | | B7.2 (% | (ng/10⁶ |
| rAAV vector | Experiment | positive cells) | cells/48 h) |
| rAAV-(B7.2/GM-CSF) | 1 (500 μl virus/6 × 10⁵ cells) | 28.5 | 25 |
| rAAV-(B7.2free/GM-CSF) | 1 (500 μl virus/6 × 10⁵ cells) | 32.3 | 27 |
| rAAV-(B7.2/GM-CSF) | 2 (5 μl virus/6 × 10⁵ cells) | 42.8 | 83 |
| rAAV-(B7.2free/GM-CSF) | 2 (2 μl virus/6 × 10⁵ cells) | 49.5 | 79 |
| rAAV-(B7.2/GM-CSF) | 2 (25 μl virus/6 × 10⁵ cells) | 68.2 | 233 |
| rAAV-(B7.2free/GM-CSF) | 2 (25 μl virus/6 × 10⁵ cells) | 71.9 | 270 |

As is evident from table 2, very large quantities of rAAV were obtained with both vector plasmids, and the quantity of rAAV obtained on use of the vector plasmid with the deletion in the C palindrome of the 5′-located ITR is additionally higher than the quantity obtained on use of the vector plasmid B7.2/GM-CSF.

3. Production of Helper Constructs

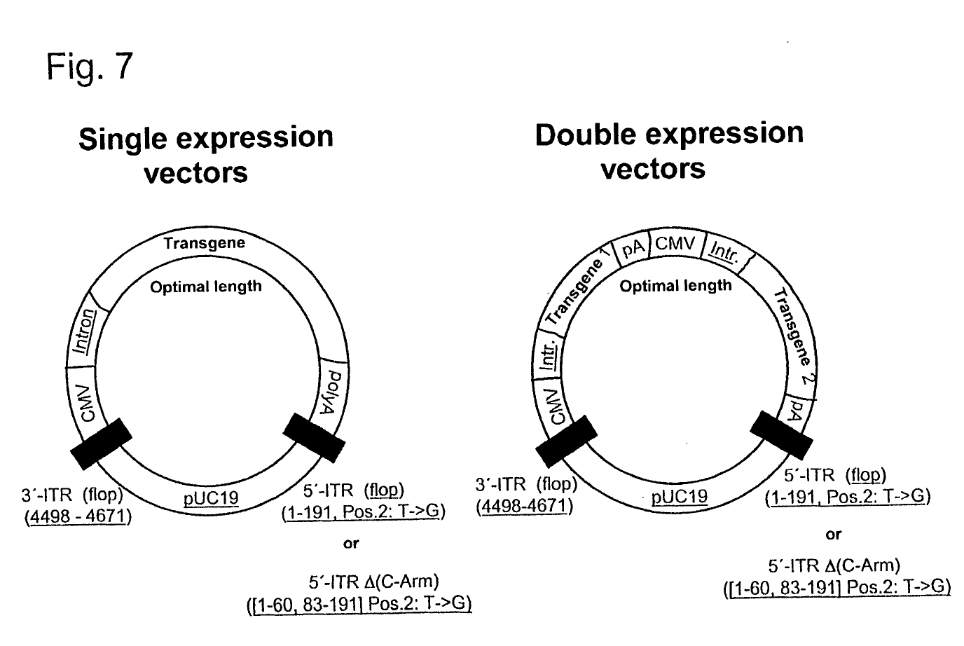

The AAV bases 190 to 1060 were amplified from wild-type AAV DNA by means of PCR. The 5′ primer for this was chosen so that a unique XbaI cleavage site was introduced at position 199. The PCR fragment was then cut with XbaI and BamHI and cloned into the vector pUC19 cut in the same way. Position 199 in the AAV genome was chosen in order to ensure that all important P5 promoter elements are present in the helper construct. After a sequencing check, wild-type AAV DNA was cut with BamHI and SnaBI, and the insert fragment was subsequently cloned into the BamHI and SmaI position of the intermediate. The basic helper construct pUC“rep/cap” was obtained in this way (FIG. 6). This helper construct comprises the AAV sequences 201 to 4497, while the vector construct harbors the AAV sequences 1 to 191 and 1-60/83-191 (left ITR) and 4498 to 4671 (right ITR) (cf. FIG. 7). It was ensured in this way that no homologous AAV sequence overlaps exist on the plasmids. Then A37 bases which are not required for optimal expression of the AAV Rep and Cap genes was deleted from the 3′ end of the AAV genome in pUC“rep/cap”. The resulting helper construct is referred to as pUC“rep/cap” Δ37 and comprises the minimalized AAV sequence from position 201 to 4460 (FIG. 6).

The Rep binding site (RBS) in the vector backbone of pUC19 (position 684 to 708 inclusive; sequence: 5′-CTCTTCCGCTTCCTCGCTCACTGAC-3) was then deleted in order to avoid non-homologous recombination at this position. The construct is named pUC“rep/cap” (RBS)Δ37 (cf. FIG. 6). In some plasmids there was additionally mutation of the three RBS in the Rep gene (P5 and P19 promoters) individually and in various combinations, without changing the amino acid sequences of the Rep protein (FIG. 6).

In a further helper construct, a so-called functional separation sequence (fs) with a length of 638 base pairs was inserted between the Rep gene and Cap gene. For this purpose, firstly the AAV sequence 1691 to 2328, which comprises the Rep C terminus and the AAV P40 promoter including Cap sequences and all regulatory sequences essential for P40, was doubled and cloned behind the stop codon for the spliced Rep versions (position 2329). This resulted in the AAV P40 promoter, which controls the cap gene, being doubled and inserted behind the rep gene. The P40 promoter was then destroyed in the rep gene without changing the Rep amino acid sequence. This was ensured by a mutation of the P40 TATA box. The overall changes for the necessary cloning steps were in the following AAV nucleotides, but without changing the amino acid sequence of Rep and Cap thereby: 1693 (T→A), 1694 (T→G), 2330 (G→C), 2331 (G→T), 2332 (G→A), 1625 (C→T), 1628 (A→G), 1826 (A→C), 1827 (A→T) and 1828 (G→C). The resulting helper construct is referred to as pUC“rep/fs/cap” Δ37 (FIG. 6).

An additional sequence was inserted into pUC“rep/cap” (RBS)Δ37 analogously, resulting in a helper construct referred to as pUC“rep/fs/cap” (RBS)Δ37 (FIG. 6).

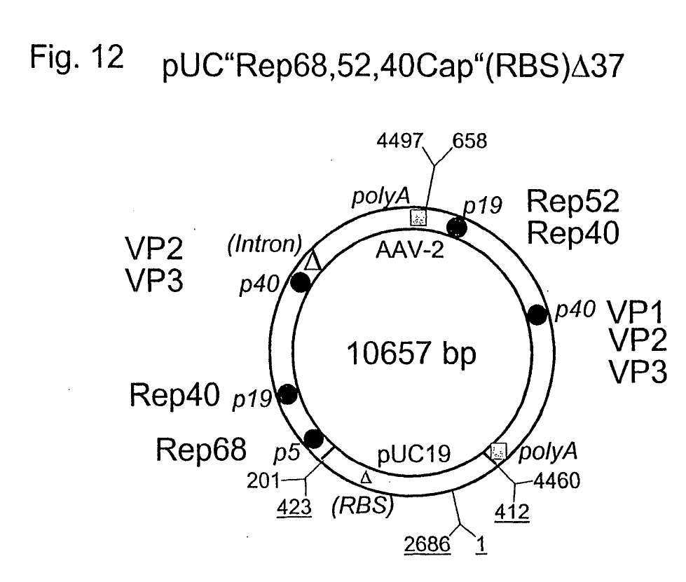

The further Rep 78-deficient helper constructs were produced by deleting the AAV nucleotides 1907 to 2227, which correspond to the rep intron, in the helper construct pUC“rep/cap” (RBS)Δ37 (cf. FIG. 6) by double-strand mutagenesis, resulting in the plasmid pUCAAVsplice as intermediate. The plasmid pUCAAVsplice was linearized with the restriction enzyme NdeI, treated with an exonuclease, for example mung bean nuclease (Boehringer Mannheim, Germany), and then mixed with the restriction enzyme SphI. The fragment with a length of 4222 bp obtained in this way was connected by ligation with a vector fragment to give the Rep 78-deficient helper construct pUC“Rep68,52,40Cap” (RBS)Δ37 (10657 bp) (cf. FIG. 12). This vector fragment can be obtained by using, for example, pUC“rep/cap” (RBS)Δ37 (cf. FIG. 6), which is obtained with a length of 6435 bp after treatment with the restriction enzymes NruI and SphI.

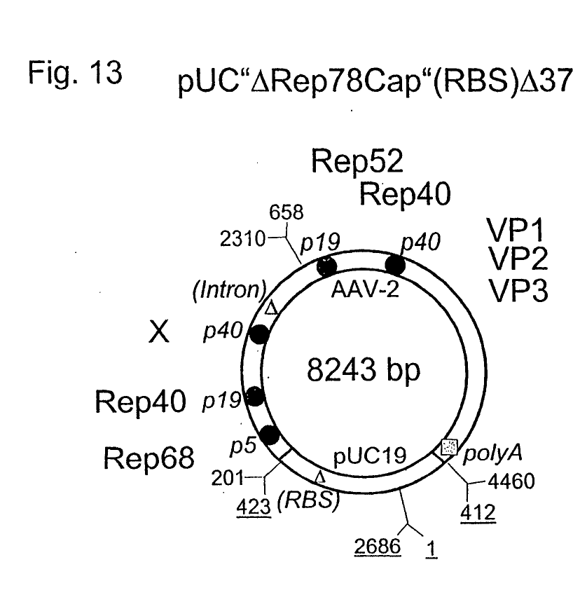

The same vector fragment can further be used to produce a further Rep 78-deficient helper construct pUC“ΔRep78Cap” (RBS)Δ37 (cf. FIG. 13). For this purpose, the intermediate pUCAAVsplice was treated with the restriction enzymes AseI, BsrBI and SphI. The BsrBI-SphI fragment with a length of 1808 bp was connected by ligation with the vector fragment with a length of 6435 bp to give said helper construct pUC“ΔRep78Cap” (RBS)Δ37 with a total length of 8243 bp. Through this cloning strategy, the rep gene in both Rep 78-deficient helper constructs was partially duplicated, so that enhanced expression of the Rep proteins Rep 68, Rep 52 and Rep 40 by both rep genes is made possible. Finally, the AAV DNA section 2946-4049 can be deleted from this construct by digestion with Apa 1 and religation, resulting in the Rep expression plasmid pUC“ΔRep78ΔCap” (RBS)Δ37, which encodes exclusively Rep 68, Rep 52 and Rep 40. The helper plasmids described above and depicted diagrammatically in FIG. 6 all have approximately comparable packaging capacities.

TABLE 3

| | Transducing rAAV- |
|---|---|
| Helper plasmid | GFP titer (tP/ml) |
| pUC“rep/cap” | 1.38 × 10⁷ |
| pUC“rep/cap” Δ37 | 1.49 × 10⁷ |
| pUC“rep/fs/cap” Δ37 | 1.26 × 10⁷ |
| pUC“rep/cap” (RBS) Δ37 | 1.46 × 10⁷ |
| pUC“rep/fs/cap” (RBS) Δ37 | 9.11 × 10⁶ |
| pUC“rep/cap” Δ37 (seq) | 2.48 × 10⁷ |

tP = transducing particles
seq = sequential transfection

Rep78-deficient helper constructs show on sequential transfection of helper construct and vector construct (experiments 1 and 3, depicted in table 4 below, and a further experiment in table 5) approximately equally good packaging efficiencies compared with Rep78-encoding helper constructs. Cotransfections were carried out in experiment 2, whereby other experimental conditions were chosen and experiment 2 can therefore not be compared with experiment 1 and 3 in relation to the packaging efficiencies either.

TABLE 4

| | Exp. 1 | Exp. 2 | Exp. 3 |
|---|---|---|---|
| | transd. titer | transd. titer | trasd. titer |
| Helper construct | (tP/ml) | (tP/ml) | (tP/ml) |
| pUC“rep/cap” Δ37 | 4.87E+06 | 7.80E+07 | 4.90E+07 |
| pUC“rep/cap” (RBS) Δ37 | 8.13E+06 | 2.60E+07 | 3.80E+07 |
| pUC“rep/fs/cap” Δ37 | 7.26E+06 | 2.50E+07 | 4.10E+07 |
| pUC“rep/fs/cap” (RBS) Δ37 | 2.74E+06 | 3.50E+06 | 5.20E+07 |
| pUC“Rep68, 52, 40Cap” (RBS) | 1.98E+06 | 2.50E+06 | 2.40E+07 |
| Δ37 | | | |
| pUC“ΔRep78Cap” (RBS) Δ37 | n.d. | 2.50E+06 | 1.80E+07 |

Cotransfection of the Rep helper constructs of the invention p5RepΔ37 (encodes all 4 Rep proteins but no Cap) and pUC“ΔRep78ΔCap” (RBS)Δ37 (codes exclusively for the Rep proteins Rep 68, 52 and 40) together with the Cap construct p5p19p40CapΔ37 in the ratio 1:1 into suitable packaging cells allows, on subsequent sequential or simultaneous transfection of a vector construct and superinfection with adenoviruses, again comparable rAAV titers to be achieved as when a single helper construct which codes for both rep and cap is transfected into the cells (pUC“rep/cap” (RBS)Δ37, pUC“Rep68,52,40Cap” (RBS) A37, pUC“Rep78Cap” (RBS)Δ37). Data on this are shown in table 5. Surprisingly, to date no rcAAV contaminants whatever have been found in rAAV lysates produced in this way.

TABLE 5

| | rAAV-GFP titer |
|---|---|
| Helper plasmid (seq) | (tP/ml) |
| pUC“rep/cap” (RBS) Δ37 | 4.64 × 10⁷ |
| pUC“Rep68, 52, 40Cap” (RBS) Δ37 | 2.19 × 10⁷ |
| pUC“ΔRep78Cap” (RBS) Δ37 | 3.13 × 10⁷ |
| p5RepΔ37 + p5p19p40CapΔ37 | 1.53 × 10⁷ |
| pUC“ΔRep78ΔCap” (RBS) Δ37 + p5p19p40CapΔ37 | 3.44 × 10⁷ |

tP = transducing particles
seq = sequential transfection

For the purposes of the transfection experiments, 4 μg of vector construct (pAAV-GFP) were contransfected with 12 μg of helper construct into 1×106 HeLa-t cells. Only in the case of the experiments designated by (seq) were initially 16 μg of helper construct and one day thereafter 16 μg of vector construct transfected sequentially. In the packaging mixtures in which rep and cap were transfected as separate helper genes (p5RepΔ37, pUC“ΔRep78ΔCap” (RBS)Δ37 and p5p19p40CapΔ37) rep and cap were cotransfected in the ratio 1:1 (that is to say 8 μg of rep and 8 μg of cap). Two days after (the first) transfection, the cells were infected with Ad-5 (MOI2). Three days later, the cells were disrupted in medium by freeze/thaw lysis, cellular constituents were pelleted, and the rAAV lysate was heat-inactivated at 60° C. for 10 min. Various dilutions of the lysate were used to infect 3×10⁵ irradiated HeLa-t cells (100 Gy). 40 hours after infection of the cells with rAAV, the cells were analyzed for GFP expression in a FACS flow (see below), and the transducing titer of the rAAV crude lysates was found therefrom. Each helper construct was tested in at least five independent experiments.

4. Optimal Rep/Cap Ratio for Triple Transfection and for Sequential Transfection

Since it is not absolutely necessary to choose a rep:cap ratio of 1:1 on use of separate rep and cap expression plasmids, the optimal rep:cap ratio was found for the following plasmid combinations.

The combinations of p5RepΔ37 with p5p19p40CapΔ37 and of pUC“ΔRep78ΔCap” (RBS)Δ37 with p5p19p40CapΔ37 were tested in each case with the vector plasmid pAAV-(B7.2free/GM-CSF). This was investigated firstly in a triple transfection (transfection of all three plasmids simultaneously), and secondly in a sequential transfection in which initially the two AAV helper plasmids and—one day later—the vector plasmid was transfected.

In the case of triple transfection of all 3 plasmids, the optimal ratio between helper plasmid to vector plasmid was always chosen as 4:1. The optimal rep:cap ratio was then determined for the helper plasmids. A rep:cap ratio of from 12:1 to 1:12 was tested for all 4 combinations. The rep:cap ratios compiled in table 6 eventually proved optimal (average of about 10 individual experiments).

TABLE 6

| | | Vector | |
|---|---|---|---|
| | | plasmid | |
| Helper plasmid | | (B7.2free/ | |
| rep plasmid | cap plasmid | GM-CSF) | rep:cap |
| p5RepΔ37 | p5p19p40CapΔ37 | triple | 1:3 |
| | | sequential | 1:7 |
| pUC“ΔRep78ΔCap” (RBS) Δ37 | p5p19p40CapΔ37 | triple | 1:3 |
| | | sequential | 1:7 |

The various plasmid combinations with in each case the optimal rep:cap ratio (see table 6) were then compared with one another in terms of their packaging efficiency. Sequential transfection of the helper plasmid pUC“rep/cap” (RBS)Δ37 with the same vector plasmid served as reference packaging (=100%). The results are compiled in table 7 as average of 10 individual experiments.

TABLE 7

| | | AdV (n010) | |
|---|---|---|---|
| | Vector | Titer | |
| Helper plasmid(s) | plasmid | (tP/ml) | % Efficiency |
| pUC“rep/cap” (RBS) Δ37 | sequential | 1.96E+07 | 100 |
| p5RepRepΔ37 + p5p19p40CapΔ37 | triple | 3.18E+07 | 162 |
| | sequential | 2.58E+07 | 132 |
| pUC“ΔRep78ΔCaP” (RBS) Δ37 + | triple | 3.09E+07 | 156 |
| p5p19p40CapΔ37 | sequential | 1.38E+07 | 70 |

It is clear from table 7 that higher packaging efficiencies can be achieved with the separate Rep-Cap helper plasmids than with the reference plasmid pUC“rep/cap” (RBS)Δ37 (132 to 162% equivalent to a factor of 1.3 to 1.6). Only the combination of pUC“ΔRep78ΔCap” (RBS)Δ37 with p5p19p40CapΔ37 in a sequential transfection with the vector plasmid shows a significant decline in particle production. (70% packaging efficiency)

It is also significant that wtAAV was not detectable in any of the 4 mixtures with the separate helper plasmids, whereas the reference plasmid still generates quantities of rcAAV in the region of 0.01% compared with the rAAV titer.

It was thus possible by means of the helper plasmids of the invention both to achieve higher packaging efficiencies and to prevent the formation of rcAAV.

5. Cell Culture

The HeLa starting cells and all packaging cell lines derived therefrom were maintained as monolayer cell cultures in Dulbecco's modified Eagle's medium (DMEM), containing 10% fetal calf serum. In addition, various selection agents were used to obtain stable cell lines from the transfected cells. In this connection, hygromycin B was used in a concentration of 500 μg/ml, neomycin in a concentration of 800 μg/ml and/or puromycin in a concentration of 1 μg/ml. The cells were cultured at 37° C. in a 5% CO₂ atmosphere. Transfection was carried out with the aid of conventional calcium phosphate precipitation techniques (Ca₃(PO₄)₂) and endotoxin-free plasmid DNA obtained using kits from Qiagen (Hilden, Germany). In order to obtain stable cell lines after cotransfection of the plasmid to be integrated and the resistance plasmid, a mixing ratio of 20:1 for the two plasmids was chosen.

6. Production of a Stable Rep-Expressing Cell Line

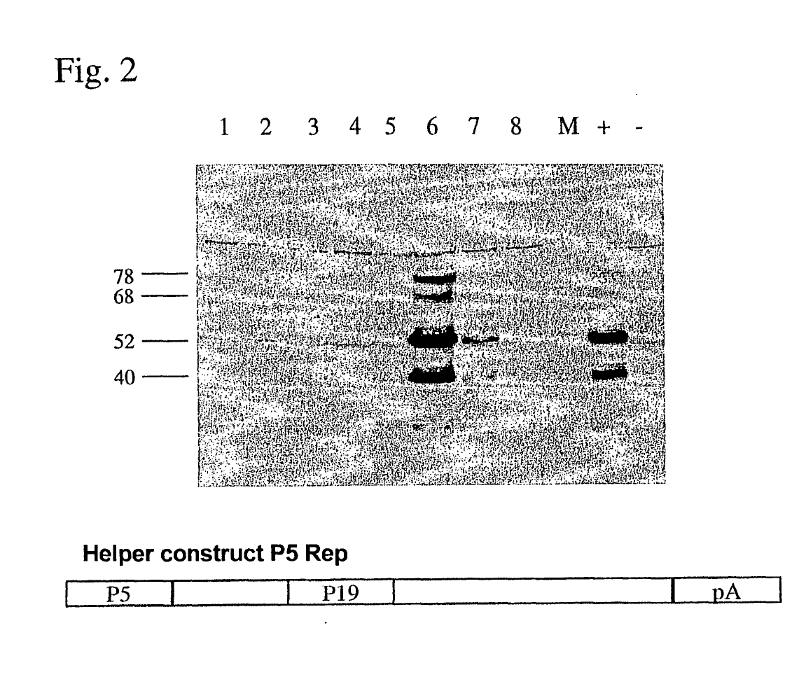

A Rep-encoding plasmid was produced by deletion of the Cap-encoding sequences from the genome of AAV2 which had no ITR sequences but all other AAV-regulatory elements (FIG. 1). Subsequently, HeLa cells were transfected with the Rep expression construct, and cell clones which expressed the Rep protein after adenovirus infection were identified by Western blotting as shown in FIG. 2. A single clone was selected and its ability to replicate an inserted rAAV genome in the presence of helper virus was characterized in detail. After identification of Rep cell line and demonstration of the functionality of the Rep proteins, this cell line was used to insert a Cap helper construct into it.

7. Identification of a Helper Construct Which was Suitable for Producing a Stable Cap Cell Line

Some plasmids in which expression of the Cap protein was controlled by the natural AAV regulatory elements were constructed. Firstly, a plasmid which comprised the cap gene under the control of the P5 promoter was constructed. This plasmid led to detectable expression of Cap protein only after helper virus infection with adenovirus, but the detectable quantities of protein were comparatively small. A second plasmid in which the DNA sequences between the P5 and the P19 promoter had been deleted was therefore constructed. This plasmid expressed not only the Cap proteins but also the P19 Rep protein. With this Cap expression plasmid, only a very small quantity of Cap protein was detectable after helper virus infection. Subsequently, a whole series of Cap plasmids which are depicted diagrammatically in FIG. 3 was constructed. All these plasmids led to a significant Cap protein expression, and the quantities of protein achieved were comparable with the quantity of protein from a plasmid containing the whole AAV genome with the exception of the ITR sequences (cf. FIG. 3 “wt”). The plasmid P5P19P40Cap as shown in FIG. 1 was used for all following experiments, because it always, in a wide variety of experiments, provided slightly larger quantities of Cap protein than the other Cap constructs (cf. FIG. 3).

8. Production of a Stable Cell Line Expressing Rep and Cap Proteins

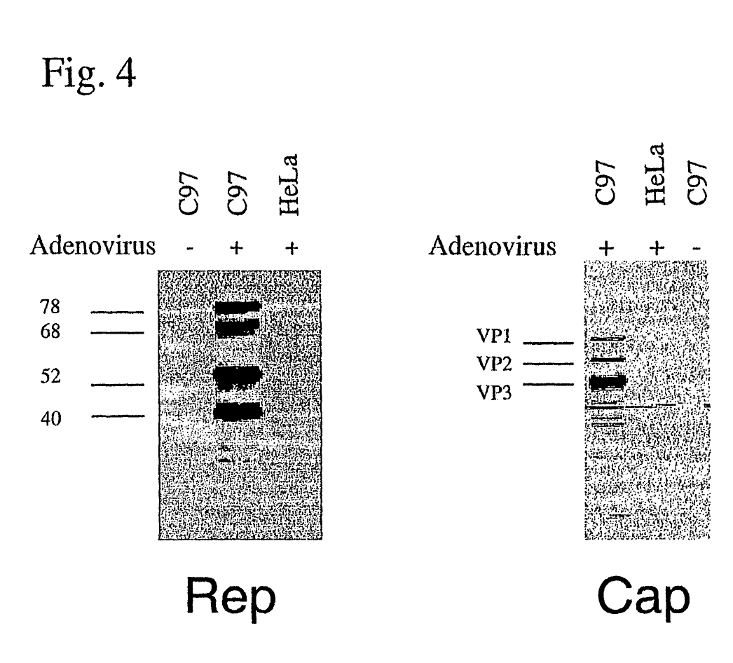

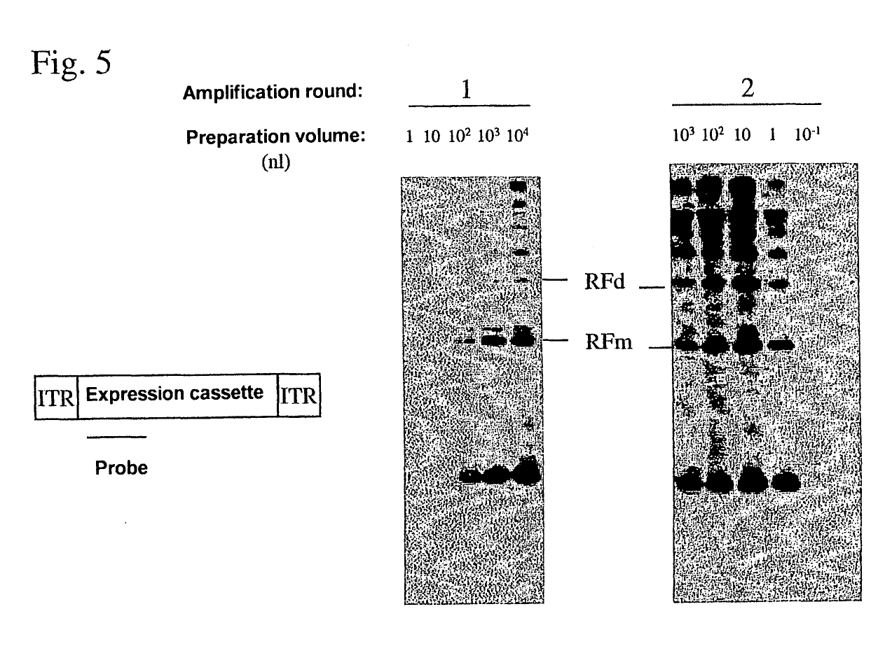

The cells line stably expressing the Rep protein and described in example 5 was subsequently transfected with the plasmid P5P19P40Cap. A cell clone which, after adenovirus infection, expressed large quantities both of Rep and of Cap proteins was then identified (cf. FIG. 4). The functionality of the Rep and Cap proteins in the selected cell line was then investigated in an infectivity experiment with two amplification rounds. The experimental results in FIG. 5 clearly show that both the Rep and the Cap proteins are functional in the selected cell line and that infectious rAAV is formed in these cells. These data additionally show that this cell line can be used to obtain an infectious titer for an rAAV stock. A Southern blotting experiment with genomic DNA proved that the Rep and Cap expression cassettes were integrated completely at two different chromosomal sites.

9. Transient Production of rAAV Using this Packaging Cell Line

Determination of how high proportion of rAAV could be achieved with this packaging cell line of the invention when a vector construct was provided in a transient transfection experiment was subsequently carried out. Vector constructs which code either for GFP or B7.2-GM-CSF (FIG. 1B) were therefore transfected into the stable cell line. 24 to 48 hours later, adenovirus was added in a multiplicity of infection of 5 (MOI 5). A viral lysate was obtained after 72 hours and was purified and used for transducing HeLa cells. It was possible to show in nine independent experiments that C97 cells are in fact able to produce rAAV when a vector plasmid and adenovirus helper function are present. The results are summarized in table 6.

TABLE 7

| Experiment No. | Vector construct | Transducing units/cell |
|---|---|---|
| 1 | GFP | 10 |
| 2 | B7.2-GMCSF | 10 |
| 3 | GFP | 5 |
| 4 | GFP | 3 |
| 5 | GFP | 4 |
| 6 | GFP | 7 |
| 7 | GFP | 10 |
| 8 | GFP | 20 |
| 9 | GFP | 1 |

A crucial advantage of the cell line of the invention is that no wild-type AAV (rcAAV) is to be expected. Numerous attempts have been made to detect rcAAV contamination, but it has not been possible to date to detect any rcAAV particles in virus stocks produced from these cell lines. This involved testing 1-2 ml of virus with about 5×10⁷ tranducing particles/ml.

10. Production of Stable Producer Cell Lines

The intention in a next step was to produce stable cell lines for producing rAAV which no longer require transfection of the vector construct for rAAV production and thus permit rAAV to be produced on a large scale. For this purpose, vector constructs were transfected into C97 cells in a plurality of independent transfection steps. Clones were initially screened after expression of the foreign DNA which were present in the rAAV genomes. Secondly, a selection was made between the clones also in relation to the virus yield due to Rep and Cap expression. After screening, 72 clones were identified for the vector construct GFP, 31 clones for the vector construct B7.2-GM-CSF and 34 clones for the vector construct nLacZ which were able to form rAAV in various orders of magnitude only after adenovirus infection. The results are shown in table 7.

TABLE 8

| | Yield | |
|---|---|---|
| Cell line | (TU/cell) | rcAAV |
| AAV-GFP | 10 | negative |
| AAV-(B7.2/GM-CSF) | 10 | negative |
| AAV-lacZ | 1 | negative |

11. Production of Further Packaging Cell Lines

The helper plasmids p5RepΔ37 (coding for all 4 Rep proteins) and pUC“ΔRep78ΔCap” (RBS)Δ37 (Rep78-deficient) described above were used in combination in each case with p5p19p40CapΔ37 (coding for the 3 capsid proteins) and a neomycin resistance gene plasmid, as described below, to produce further packaging cell lines for AAV-2 vectors.

HeLa-t cells were cotransfected at about 10% confluence with one each of rep, cap and resistance gene plasmid (in the ratio 10:10:1) and cultivated further until the cells had reached almost 100% confluence. Antibiotic selection (G418 was started 48 h after the transfection. After 100% cell confluence was reached, the cells were trypsinized and seeded in 10 cell pools each of 10% confluence. These cell pools were cultivated further for about 4 weeks (with the cells being passaged each time after 100% confluence was reached) before a first packaging experiment was started.

Replicates were produced from each pool. One replicate was cultivated further and the relevant replicate was transfected at 10% confluence with an AAV-GFP vector plasmid and, 24 h later, infected with AdV. 72 h later, the rAAV vectors were harvested and titrated as described in the examples “production of rAAV” and “virus titration”, number 12 and 13. The most efficient of the 10 pools in relation to packaging of rAAV vectors was identified in this way.

1 pool for each of the two new packaging cell lines (with and without Rep78 gene) was subsequently cloned further. Initially, about 250 pools of 100-1 000 cells were screened in packaging experiments. The pool which was again best was cloned further, analyzing in the next step about 1 000 pools of 10 cells (per cell line). The best pool thereof was finally subjected to a single cell cloning. About 100 single cell clones per packaging cell line were analyzed.

Finally, 3 single cell clones of the Rep78-encoding cell line (named SH) and 4 single cell clones of the Rep78-deficient packaging cell line (DJ) crystallized out. These clones were cultivated for a further 40 passages in the absence of G418 and thus without further selection pressure. Packaging experiments were carried out as described previously with the 7 clones about every 2 weeks. The vector plasmid used was pAAV-GFP or pAAV-(B7.2/GM-CSF). Only AdV was employed as helper virus in this series of experiments. It emerged from this that all 7 clones retained their packaging efficiency throughout the cultivation time, and it can therefore be assumed that there is stable integration of the AAV genes. All 7 clones achieved approximately comparable packaging efficiencies in the region of 1×10⁷ tP/ml on use of adenoviruses as helpers.

12. Production of rAAV

In order to produce rAAV, packaging cells were transfected with a vector plasmid and, 48 h later, infected with adenovirus (MOI 5). Producer cell lines were infected only with adenovirus. 72 hours after the adenovirus infection, the cells and the supernatant were harvested, the cells being spun down by centrifugation at 1 500 rpm for 5 min, and about 10⁷ cells/ml being resuspended in part of the supernatant. The remaining part of the supernatant was treated at 560C for 30 min and then frozen until used. The cell suspension was subjected to a “freeze/thaw” cycle three times, the freezing operation taking place at −80° C. and the thawing operation at 37° C. Cell detritus was then removed by centrifugation in a microfuge at full power for 5 min. Remaining adenovirus was inactivated by incubation at 56° C. for 30 min. The supernatant resulting from this was used for the purposes of the present invention as virus stock for further purifications.

13. Virus Titration

The virus stock always reached a titer which was functionally adequate for the transduction. HeLa cells were treated with UV light (35 J/m²) in a Stratalinker™ (can usually be purchased from Stragene Deutschland) in phosphate-buffered saline. Immediately after the irradiation, the cells were exposed to the rAAV suspension. Different volumes of a given preparation were always used in order to obtain different proportionate amounts of transduced cells. Starting from the number of target cells known therefrom, the number of transducing units was found by calculation. The proportion of transduced cells no longer showed a linear behavior at high transduction rates, but was approximately linear when transduction rates below 10% were chosen. The titrations were carried out in 12-well plates, infecting 2×10⁵ HeLa cells per well. These infections were carried out in 500 μl of buffer.

In addition, some virus stocks used for the purposes of this invention were titrated using a special infection test, wherein two packaging cell amplification rounds were carried out in order to find the infectious titers of the preparations. Details of this test can be referred to, for example, in Clark et al. (1996) Gene Therapy 3, 1124-1232. For this purpose, the virus stocks were serially diluted in serum-free medium, and then portions containing known volumes of the initial virus stock were used to transduce the packaging cell line C97 for the purposes of this invention together with a constant quantity of adenovirus (MOI5). After 72 hours, the cells and the supernatant were harvested, subjected to a “freeze/thaw” cycle three times, spun down in order to remove parts of cells, treated at 56° C. in order to inactivate adenovirus, and then put on a cell culture dish with fresh C97 cells together with fresh adenoviruses (MOIS). After a further three days, the cells were harvested and their DNA was isolated by means of Hirt lysis using a standard protocol (Hirt (1967) J. Mol. Biol. 26, 365-369). The DNA was fractionated according to the size thereof in an agarose gel, blotted and incubated with a probe which contained either part of the vector construct for identification of the rAAV replication forms or a part of the wild-type AAV genome in order to identify the Rep- and Cap-containing infectious particles.

Further titrations of the vector preparations were carried out to determine the genomic titers. The resulting virus suspension was treated with DNAseI and then incubated with addition of 5 mM EDTA at 68° C. for 30 min, and DNA entrapped in capsid was released by a subsequent incubation with proteinaseK (0.5 mg/ml) in 0.5% SDS. The DNA was then purified by phenol/chloroform extraction, subsequently subjected to an ethanol precipitation and blotted onto a nylon membrane using a dot-blot apparatus, and then hybridized with probes which were specific for rAAV or wild-type AAV. The ratio between genomic particles and infectious particles was 100 to 1 000:1, depending in each case on the preparation investigated.

The helper virus adenovirus type 5, which was used for the purposes of the present invention, was obtained by plaque purification. After replication in HeLa cells and purification by double CsCl gradient centrifugation, adenovirus was again titrated on HeLa cells in order to obtain the required number of plaque-forming units (PFU) per ml (PFU/ml). Before use, a small portion of the resulting virus stock was tested for the presence of wild-type AAV, but this was always negative.

14. Protein and DNA Analysis

The low molecular weight DNA and the genomic DNA were isolated using standard isolation methods (Sambrook et al. (1989) supra; Hirt (1967) supra). For Southern blotting analyses of genomic DNA, 15 μg of DNA were mixed with the required restriction enzyme, the mixture was then fractionated in an agarose gel, and the DNA was transferred to a nylon membrane using a vacuum blotter (from Pharmacia, Erlangen, Germany). When DNA obtained by Hirt lysis was used, an amount corresponding to 10⁵ cells was loaded on each lane. The probes used for the AAV genomes were a 1.5 kb HincII cap-specific fragment or a 655 bp BamHI-BstEII rep-specific fragment. The probe used for the rAAV genomes which were used for the purposes of the present invention was a sample corresponding to the CMV promoter. The probes were labeled with digoxigenin, which can usually be obtained as kit from Boehringer Mannheim, Germany, and was used in accordance with the manufacturer's instructions. An alkaline phosphatase-conjugated anti-digoxigenin antibody was used for the detection, followed by incubation with the substrate CDP Star™ and an autoradiography. For dot-blot experiments, the samples were adjusted to a final concentration of 0.4 N NaOH and transferred to a membrane using the aforementioned vacuum blotter.

For protein analyses, the samples were resuspended in Laemli sample buffer comprising 100 mM DTT, and then boiled for 15 min before they were analyzed in an SDS-PAGE. The proteins were then blotted using either a liquid transfer system (from Sigma, Germany) or a semi-dry system (from Hofer, Germany). Detection took place with the Rep-specific antibody 303.9 and with the Cap-specific antibody Bl (Wistuba et al. (1997) J. Virol. 71, 1341-1353).

15. FACS Analyses

B7.2 expression was investigated using a commercially available FITC-conjugated antibody directed against B7.2 (from Pharmingen International, USA). In order to preclude nonspecific staining, an isotype control antibody and staining of mock-transduced cells with a B7.2 antibody was carried out. The cells were incubated with the antibody on ice for 30 min and then incubated twice in phosphate-buffered saline before they were analyzed. The antibody staining was carried out in D10 buffer, and the cells were resuspended after the washing steps in D10 buffer and subjected to the FACS analysis. The fluorescence was carried out using a Beckton Dickinson FACS vantage flow cytometer at an extinction wavelength of 488 nm and an emission wavelength of 530+15 nm. The proportion of positive cells was defined as the portion whose fluorescence intensity was greater than 99% of the control cells.

1. A host cell for packaging recombinant adeno-associated virus (rAAV) comprising at least one copy of a helper construct for expression of at least one AAV rep protein and at least one AAV Cap protein, characterized in that the nucleic acids coding for the Rep protein and the Cap protein are functionally separate and are operatively linked to the natural AAV regulatory sequences, in particular, to the natural AAV promoters.

2. The host cell as claimed in claim 1, characterized in that the recombinant adeno-associated virus (rAAV) is selected from the serotypes AAV1, AAV2, AAV3, AAV4, AAV5, AAV6 and/or from a capsid mutant of AAV.

3. The host cell as claimed in claim 1 or 2, characterized in that it additionally comprises at least one copy of a vector construct (producer cell).

4. The host cell as claimed in any of claims 1 to 3, characterized in that it comprises at least one copy of a nucleic acid construct for at least one gene product of a helper virus and/or of a cellular gene which is necessary for the production of rAAV.

5. The host cell as claimed in any of claims 1 to 4, characterized in that the nucleic acids coding for the Rep protein and the Cap protein are located on two different helper constructs.

6. The host cell as claimed in any of claims 1 to 5, characterized in that the helper constructs for expression of the Rep protein and of the Cap protein are integrated at two different sites into the genome of the host cell.

7. The host cell as claimed in any of claims 1 to 6, characterized in that expression of the Rep protein is controlled by the natural AAV promoter P5.

8. The host cell as claimed in any of claims 1 to 7, characterized in that expression of the Cap protein is controlled by the natural AAV promoter P40, in particular by the natural AAV promoters P19 and P40, especially by the natural AAV promoters P5, P19 and P40.

9. The host cell as claimed in any of claims 1 to 8, characterized in that expression of the rep protein and of the cap protein in the host cell is regulated dependent on one another.

10. The host cell as claimed in any of claims 1 to 9, characterized in that termination of the transcription of the nucleic acids coding for the Rep protein and the Cap protein is controlled by the natural regulatory sequences, in particular by the natural AAV poly-A signal.

11. The host cell as claimed in any of claims 1 to 10, characterized in that the host cell is a mammalian cell, in particular a human cervical carcinoma cell, especially an HeLa cell.

12. A helper construct for the expression of at least one AAV Rep protein in a host cell, characterized in that the nucleic acid coding for the Rep protein is operatively linked to the natural regulatory sequences of AAV, in particular to the natural AAV promoter P5.

13. The helper construct as claimed in claim 12 comprising nucleic acid sequences coding for at least one Rep protein, characterized in that the Rep proteins are Rep 68, Rep 52 and/or Rep 40, but not Rep 78.

14. A helper construct for expression of at least one AAV Cap protein in a host cell, characterized in that the nucleic acid coding for the Cap protein is operatively linked to the natural regulatory sequences of AAV, preferably to the natural AAV promoter P40, in particular to the natural AAV promoters P19 and P40, especially to the natural AAV promoters P5, P19 and P40.

15. A vector construct comprising one or more nucleic acid(s) which are heterologous to AAV and are flanked by ITR sequences, characterized in that the 5′-located ITR sequence has a deletion in the region of the C palindrome.

16. The vector construct as claimed in claim 15, characterized in that the deletion of the 5′-located ITR sequence amounts to 150 nucleotides, preferably 80 nucleotides, in particular 40 nucleotides, especially 22 nucleotides, of the C palindrome.

17. A vector construct comprising one or more nucleic acids heterologous to AAV, in particular a nucleic acid coding for a protein selected from a cytokine, in particular IL2, IL4, IL12 and/or GM-CSF and/or a costimulating molecule, in particular B7, especially B7.1 and/or B7.2, which are flanked in particular by AAV ITR sequences, and expression of the heterologous nucleic acid(s) are controlled by a promotor and/or enhancer which are heterologous to AAV, in particular by CMV MIEP.

18. A method for producing a host cell for the packaging and/or production of recombinant adeno-associated virus (rAAV), which comprises the steps:
(a) production of a Rep helper construct as claimed in claim 11 or 12 and of a Cap helper construct as claimed in claim 13,
(b) introduction of the Rep helper construct into a host cell,
(c) selection of a host cell comprising the Rep helper construct,
(d) introduction of the Cap helper construct into the selected host cell from step (c) and
(e) selection of a host cell comprising the Rep helper construct and the Cap helper construct.

19. The method as claimed in claim 18, characterized in that the Cap helper construct is introduced into the host cell before the Rep helper construct.

20. The method as claimed in claim 18, characterized in that the Rep helper construct and the Cap helper construct are introduced into the host cell at essentially the same time.

21. The method as claimed in any of claims 18 to 20, characterized in that at least one Cap helper construct, at least one Rep helper construct, at least one vector construct and/or at least one helper gene is introduced into the host cell.

22. The method as claimed in any of claims 18 to 21, characterized in that the constructs and/or genes are transfected, in particular stably transfected.

23. The method as claimed in any of claims 18 to 21, characterized in that the constructs and/or genes are introduced with viruses, in particular with recombinant viruses, especially rAAV, adenoviruses, herpesviruses, vacciniaviruses baculoviruses, phages and/or bacteriophages, into the host cell.

24. The method as claimed in any of claims 18 to 23, characterized in that a preselection for cells with integration events is carried out, in particular by joint introduction of a reporter construct with one of the constructs and/or genes into a host cell and subsequent selection for the reporter.

25. The method as claimed in any of claims 18 to 24, characterized in that selection of the construct- and/or gene-containing host cell takes place by detection of protein expression, in particular by Western blotting.

26. The method as claimed in any of claims 18 to 24, characterized in that selection of the construct- and/or gene-containing host cell takes place by detection of specific nucleic acids, in particular by Southern blotting, Northern blotting and/or quantitative PCR.

27. The method as claimed in any of claims 18 to 24, characterized in that selection of the construct- and/or gene-containing host cell takes place by detection of rAAV particle formation, in which the constructs lacking for rAAV particle formation are introduced into the host cell and/or are infected with helper viruses.

28. The use of a host cell as claimed in any of claims 1 to 11 and/or of at least one Rep helper construct as claimed in claim 12 or 13 and of at least one Cap helper construct as claimed in claim 14 and/or of a vector construct as claimed in any of claims 15 to 17 for producing rAAV.

29. The use of rAAV as claimed in claim 28 for transferring genes into cells, in particular immuno-stimulatory genes, especially GM-CSF, B7.1 and/or B7.2.


SEQUENCE LISTING (6 sequences)

SEQ ID NO: 1 - DNA, 25 bp, Artificial Sequence
  pUC19 Rep binding site
ctcttccgcttcctcgctcactgac

SEQ ID NO: 2 - DNA, 63 bp, AAV
tcactgaggccgggcgaccaaaggtcgcccgacgcccgggctttgcccgggcggcctcagtga

SEQ ID NO: 3 - DNA, 63 bp, AAV
agtgactccggcccgctggtttccagcgggctgcgggcccgaaacgggcccgccggagtcact

SEQ ID NO: 4 - DNA, 191 bp, AAV
tgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacgtgaattacgtcatagggttagggagg

SEQ ID NO: 5 - DNA, 169 bp, AAV
tgggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacgtgaattacgtcatagggttagggagg

SEQ ID NO: 6 - DNA, 174 bp, AAV
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggga